(19) United States
(12) Patent Application Publication
(10) Pub. No.: US 2009/0286242 A1
(43) Pub. Date: Nov. 19, 2009

(54) MicroRNA Expression Profiling and Uses Thereof

(75) Inventors: Gregory J. HANNON, Huntington, NY (US); Ingrid Ibarra, Centereach, NY (US)

Correspondence Address: WilmerHale/Cold Spring Harbor Laboratory, 399 Park Avenue, New York, NY, 10022, US

(73) Assignee: COLD SPRING HARBOR LABORATORY, Cold Spring Harbor, NY (US)

(21) Appl. No.: 12/331,832

(22) Filed: Dec. 10, 2008

Related U.S. Application Data
(60) Provisional application No. 61/007,010, filed on Dec. 10, 2007.
(60) Provisional application No. 61/007,754, filed on Dec. 13, 2007.

(51) Int. Cl. C12Q 1/68

(52) U.S. Cl. 435/6

(57) ABSTRACT

Provided are methods and reagents for obtaining microRNA expression profiles in selected cell populations or sub-populations, such as stem cell or progenitor cell populations, and using such microRNA expression profiles for cell characterization, isolation/purification, and/or reinforcement of cell fate specification, both in research & development, and in therapeutic applications. Also provided are methods of identifying and isolating mammary progenitor cells using miRNA sensor constructs.

### CROSS-REFERENCE TO RELATED APPLICATIONS

This application claims the benefit of priority of U.S. Provisional Application Ser. Nos. 61/007,010, filed Dec. 10, 2007, and 61/007,754, filed Dec. 13, 2007, the disclosures of which are hereby incorporated by reference in their entirety.

### BACKGROUND OF THE INVENTION

MicroRNAs (miRNAs) are a large class of small non-coding RNAs that regulate protein expression in eukaryotic cells. Initially believed to be unique to the nematode Caenorhabditis elegans, miRNAs are now recognized to be important gene regulatory elements in multicellular organisms including plants and animals.

The majority of human miRNA loci is located within intronic regions and is transcribed by RNA polymerase II as part of their hosting transcription units. Genes encoding miRNAs are transcribed as long primary transcripts (pri-miRNAs) that are sequentially processed by components of the nucleus and cytoplasm to yield a mature miRNA.

Two members of the ribonuclease (RNase) III endonuclease protein family, Drosha and Dicer, have been implicated in this two-step processing. The primary transcripts are cleaved by Drosha to release approximately 70 nt precursor-miRNAs that form characteristic stem loop structures and are subsequently processed by Dicer to generate mature miRNAs of about 22 nt length. miRNAs are estimated to account for >3% of all human genes and to control the expression of thousands of target mRNAs, with multiple miRNAs targeting each mRNA and each miRNA having thousands of potential targets.

There are approximately 500 known mammalian miRNA genes, and each miRNA may regulate hundreds of different protein-coding genes. Mature miRNAs bind to target mRNAs in a protein complex known as the miRNA-induced silencing complex (miRISC), sometimes referred to as the miRNP (miRNA-containing ribonucleoprotein particles), where mRNA translation is inhibited or mRNA is degraded. Recent studies have indeed demonstrated that miRNAs are involved in critical biological processes by suppressing the translation of protein coding genes, and have linked the expression of selected miRNAs to carcinogenesis and viral pathogenesis.

Analysis of mutations in key RNAi components also yields insights into miRNAs function. Dicer-mutant mice die early in development with a loss of Oct4-positive multipotent stem cells (Bernstein et al., 2003). Even in the presence of a strong differentiation inducer, DGCR8/pasha knock-out ES cells fail to inactivate self-renewal programs (Wang et al, 2007). In Drosophila ovaries, dcr-1 mutant germ line stem cells are depleted within 3 weeks of Dicer loss (Jin et al., 2007), and homozygous mutation of loqs, an obligate Dicer partner, causes defects in egg chamber development (Forstemann et al., 2005; Jiang et al., 2005).

### SUMMARY OF THE INVENTION

One aspect of the invention provides a method for isolating mammary progenitor cells, comprising isolating, from a population of candidate cells, cells that preferentially express aldehyde dehydrogenase (ALDH).

In certain embodiments, the cells that preferentially express ALDH further preferentially express Stem Cell Antigen (Sca-1).

In a related aspect, the invention provides a method for isolating mammary progenitor cells, comprising contacting a population of candidate cells with an agent that preferentially eliminates cells having low ALDH activity.

In certain embodiments, the mammary progenitor cells are capable of: (1) reconstituting a functional mammary gland upon transplantation of a sufficient amount of said mammary progenitor cell into a host; (2) self-renewal; (3) differentiation into both myoepithelial and luminal cells in vitro and/or in vivo, and/or, (4) population expansion and/or exhibiting increased mammosphere forming capacity upon enforced expression of β-catenin or Wnt-1.

In certain embodiments, the population of candidate cells is from a mammary epithelial cell line or non-adherent mammosphere.

In certain embodiments, the mammary epithelial cell line is Comma-Dβ.

In certain embodiments, step (2) or (3) is determined by one or more of: 2-D culture, 3-D culture, mammosphere assay, in vivo morphogenic potential assay, and colony formation assay.

In certain embodiments, the cells that preferentially express ALDH and/or Sca-1 are isolated by comparing ALDH activity in the presence or absence of an ALDH inhibitor.

In certain embodiments, the ALDH inhibitor is DEAB.

In certain embodiments, the cells that preferentially express ALDH and/or Sca-1 constitute no more than 3% of said population of candidate cells.

In certain embodiments, the agent is an oxazaphosphorine.

In certain embodiments, the agent is mafosfamide (MAF).

Another aspect of the invention provides a method for determining a microRNA (miRNA) expression profile of a population of mammary progenitor cells, the method comprising: (1) obtaining the population of mammary progenitor cells and a control population of cells; (2) obtaining miRNA expression profiles for the population of mammary progenitor cells and the control population of cells; (3) comparing the miRNA expression profile for the population of mammary progenitor cells with that of the control population of cells, and, (4) identifying one or more miRNA that is expressed at a statistically significantly higher or lower level in the population of mammary progenitor cells compared to the control population of cells, thereby determining the miRNA expression profile of the population of mammary progenitor cells.

In certain embodiments, the population of mammary progenitor cells is progenitor cells from normal/healthy tissue.

In certain embodiments, the mammary progenitor cells preferentially express ALDH and/or Sca-1.

In certain embodiments, the mammary progenitor cells are obtained by any of the subject methods.

In certain embodiments, the control population of cells expresses low level of Sca-1 or no detectable level of Sca-1.

In certain embodiments, the miRNA expression profiles are determined by using miRNA microarray, deep sequencing analysis, and/or quantitative stem-loop PCR (qRT-PCR).

In certain embodiments, the population of mammary progenitor cells is tumor progenitor cells, and the control population of cells is matched healthy cells.

Another aspect of the invention provides a method of screening for a drug useful for cancer treatment, comprising: (1) contacting tumor progenitor cells with a candidate compound; (2) determining whether the candidate compound inhibits proliferation and/or survival, or promotes benign differentiation of the tumor progenitor cells; wherein an observed inhibition of proliferation and/or survival, and/or enhanced benign differentiation of the tumor progenitor cells is indicative that the candidate compound is potentially useful as the drug for cancer treatment.

Another aspect of the invention provides an isolated mammary progenitor cell that preferentially expresses ALDH and/or Sca-1.

Another aspect of the invention provides an isolated mammary progenitor cell that preferentially express miR-205 and/or miR-22.

Another aspect of the invention provides an isolated mammary progenitor cell that substantially lacks expression of let-7b, let-7c, and/or miR-93.

Another aspect of the invention provides a method of isolating mammary progenitor cells, comprising isolating, from a population of candidate cells, cells that preferentially express miR-205 and/or miR-22, or cells that substantially lack expression of let-7b, let-7c, and/or miR-93.

In certain embodiments, the cells that preferentially express miR-205 and/or miR-22 are isolated by: (1) introducing into the population of candidate cells an miRNA sensor that detects the presence of miR-205 and/or miR-22 by eliminating the expression of a marker; and, (2) isolating cells that do not express the marker.

In certain embodiments, the method further comprises enforcing expression of miR-205 and/or miR-22 in the population of candidate cells before step (2).

In certain embodiments, the cells that substantially lack expression of let-7b, let-7c, and/or miR-93 are isolated by: (1) introducing into the population of candidate cells an miRNA sensor that detects the presence of let-7b, let-7c, and/or miR-93 by eliminating the expression of a marker; and, (2) isolating cells that express the marker.

In certain embodiments, the miRNA sensor comprises: (1) a first polynucleotide sequence complementary to the sequence of one or more of miR-205, miR-22, let-7b, let-7c, or miR-93; (2) a second polynucleotide sequence encoding the marker; wherein the presence of miR-205, miR-22, let-7b, let-7c, and/or miR-93 inhibits the expression of the marker.

In certain embodiments, the first polynucleotide and the second polynucleotide form a transcription unit, and the transcription product of the transcription unit is targeted for destruction by an RNAi mechanism in the presence of miR-205, miR-22, let-7b, let-7c, and/or miR-93.

In certain embodiments, the marker encodes an enzyme or a fluorescent protein.

In certain embodiments, the fluorescent protein is DsRed or GFP, or a mutant thereof with a shifted emission maximum.

An additional aspect of the invention provides a method of isolating mammary progenitor cells from a population of mammary cells in culture, the method comprising a) introducing into the population of mammary cells an expression cassette comprising (i) a first nucleotide sequence encoding a reporter, and (ii) a second nucleotide sequence complementary to about 12-25 contiguous nucleotides of let-7b, let-7c, or miR-93, wherein the presence of let-7b, let-7c, or miR-93 in a cell inhibits expression of the reporter in the cell; and, b) isolating cells that do not express the reporter; thereby isolating mammary progenitor cells.

A further aspect of the invention provides a method of isolating mammary progenitor cells from a population of mammary cells in culture, the method comprising a) introducing into the population of mammary cells an expression cassette comprising (i) a first nucleotide sequence encoding a reporter, and (ii) a second nucleotide sequence complementary to about 12-25 contiguous nucleotides of miR-205 or miR-22 in a cell inhibits expression of the reporter in the cell, wherein the presence of miR-205 or miR-22 in a cell inhibits expression of the reporter in the cell; and, b) isolating cells that express the reporter; thereby isolating mammary progenitor cells.

In some embodiments, the population of mammary cells is from a mammary epithelial cell line or a non-adherent mammosphere.

In some embodiments, the expression cassette is introduced by transfection, whereas in other embodiments, the expression cassette is introduced by infection. Where infection is used, the expression cassette can further comprise a 5′ LTR, a 3′ LTR, and a viral packaging signal.

The reporter is can be a fluorescent protein, a toxin, or any other marker discussed herein or known in the art.

Preferably, the second nucleotide sequence is at least 19 nucleotides in length. Preferably, the second nucleotide sequence is located in an untranslated region (UTR) of the first nucleotide sequence. In another preferably embodiment, the second nucleotide sequence is located in the 3′ UTR of the sequence encoding the reporter.

In one embodiment, the second nucleotide sequence is perfectly complementary to miR-205, miR-22, let-7b, let-7c, or miR-93. In another embodiment, the complementarity is imperfect. The expression cassette can comprise a nucleotide sequence complementary to about 12 to 23 contiguous nucleotides of at least two miRNAs selected from the group consisting of miR-205, miR-22, let-7b, let-7c, and miR-93.

Another aspect of the invention provides an miRNA sensor for sensing the presence of a target miRNA, comprising: (1) a first polynucleotide sequence complementary to the sequence of the target miRNA; (2) a second polynucleotide sequence encoding a fluorescent marker or a toxin marker; wherein the presence of the target miRNA inhibits the expression of the fluorescent marker or the toxin marker.

In certain embodiments, the first polynucleotide and the second polynucleotide form a transcription unit, and the transcription product of the transcription unit is targeted for destruction by an RNAi mechanism in the presence of the target miRNA.

In certain embodiments, the fluorescent marker is DsRed or GFP, or a mutant thereof with a shifted emission maximum.

In certain embodiments, the first polynucleotide is partially complementary to the sequence of the target miRNA.

In certain embodiments, the first polynucleotide is further complementary to the sequence of a second target miRNA.

In certain embodiments, the first polynucleotide is located at the 3′-UTR region of the second polynucleotide sequence encoding the fluorescent marker or the toxin marker.

In another aspect, the invention provides a method of identifying mammary progenitor cells in a population of mammary cells, the method comprising a) introducing into the population of mammary cells an expression cassette comprising (i) a first nucleotide sequence encoding a reporter, and (ii) a second nucleotide sequence complementary to about 12-25 contiguous nucleotides of let-7b, let-7c, or miR-93, wherein the presence of let-7b, let-7c, or miR-93 in a cell inhibits expression of the reporter in the cell; and, b) identifying cells that do not express the reporter; thereby identifying mammary progenitor cells.

In a further aspect, the invention provides a method of identifying mammary progenitor cells in a population of mammary cells, the method comprising a) introducing into the population of mammary cells an expression cassette comprising (i) a first nucleotide sequence encoding a reporter, and (ii) a second nucleotide sequence complementary to about 12-25 contiguous nucleotides of miR-205 or miR-22 in a cell inhibits expression of the reporter in the cell, wherein the presence of miR-205 or miR-22 in a cell inhibits expression of the reporter in the cell; and, b) identifying cells that do not express the reporter; thereby identifying mammary progenitor cells.

The expression cassette can comprise a tissue-specific promoter, a developmental stage specific promoter, or an inducible promoter.

Cells not expressing the reporter are identified using techniques described herein and known in the art, for example, a luminometer.

Another aspect of the invention provides a method of identifying or isolating, from a population of candidate cells, a subpopulation of cells that preferentially express a target miRNA, the method comprising: (1) introducing into the population of candidate cells an miRNA sensor that detects the presence of the target miRNA by eliminating the expression of a marker; and, (2) isolating cells that do not express the marker.

Another aspect of the invention provides a method of identifying or isolating, from a population of candidate cells, a subpopulation of cells that substantially lack expression of a target miRNA, the method comprising: (1) introducing into the population of candidate cells an miRNA sensor that detects the presence of the target miRNA by eliminating the expression of a marker; and, (2) isolating cells that express the marker.

In certain embodiments, the subpopulation of cells comprises no more than 1% of the population of candidate cells.

In certain embodiments, the subpopulation of cells are enriched at least about 100-fold from the population of candidate cells.

In certain embodiments, the method of further comprises introducing into the population of candidate cells a second miRNA sensor that detects the presence of a second target miRNA by eliminating the expression of a second marker.

In certain embodiments, the second marker is the same as said marker.

In certain embodiments, the second marker is different from said marker.

In certain embodiments, the second marker can be used in conjunction with said marker.

Another aspect of the invention provides a method of deleting, from a population of candidate cells, a subpopulation of cells that preferentially express a target miRNA, the method comprising: (1) introducing into the population of candidate cells an miRNA sensor that detects the presence of the target miRNA by eliminating the expression of a marker; and, (2) eliminating/deleting cells that do not express the marker.

Another aspect of the invention provides a method of deleting, from a population of candidate cells, a subpopulation of cells that substantially lack expression of a target miRNA, the method comprising: (1) introducing into the population of candidate cells an miRNA sensor that detects the presence of the target miRNA by eliminating the expression of a marker; and, (2) eliminating/deleting cells that express the marker.

In certain embodiments, the subpopulation of cells is tumor progenitor cells.

In certain embodiments, the marker is a toxin, and wherein the subpopulation of cells is tumor progenitor cells that lack the expression of the target miRNA.

Another aspect of the invention provides a method for expanding a subpopulation of mammary progenitor cells in a population of mammary epithelial cells comprising said mammary progenitor cells, the method comprising enforcing expression of miR-205 and/or miR-22, and/or inhibiting expression of let-7b, let-7c, and/or miR-93.

In certain embodiments, the expression of let-7b, let-7c, and/or miR-93 is inhibited by an antagomir that competitively inhibits RISC by binding to let-7b, let-7c, and/or miR-93, respectively.

In certain embodiments, the expression of let-7b, let-7c, and/or miR-93 is inhibited by inhibiting transcriptional or post-transcriptional processing of a precursor molecule for let-7b, let-7c, and/or miR-93, respectively.

In certain embodiments, the mammary epithelial cells are Comma-Dβ cells.

Another aspect of the invention provides a method for dedifferentiating a differentiated cell, comprising inhibiting the expression of let-7b, let-7c, and/or miR-93 in the differentiated cell.

In certain embodiments, the differentiated cell is reverted back to exhibit at least one progenitor/stem cell phenotype after the expression of let-7b, let-7c, and/or miR-93 is inhibited.

Another aspect of the invention provides a method for regulating the state of differentiation of a normal, untransformed cell, comprising introducing an antagomir nucleic acid into the cell, which antagomir inhibits a microRNA that regulates one or more of differentiation or proliferation of the cell.

Another aspect of the invention provides a method for inducing dedifferentiation, comprising contacting a differentiated cell with an antagomir nucleic acid that inhibits an antiproliferative microRNA.

Another aspect of the invention provides a method for maintaining pluripotency of a stem cell, comprising contacting the stem cell with an antagomir nucleic acid that inhibits an antiproliferative microRNA.

In certain embodiments, the antiproliferative microRNA is a let-7 miRNA, such as let-7c miRNA.

In certain embodiments, the antagomir nucleic acid is transcribed from a vector introduced into the stem cell.

In certain embodiments, the antagomir nucleic acid is ectopically contacted with the stem cell, and is taken up thereby.

In certain embodiments, the antagomir comprises a sequence that is substantially complementary to 12 to 23 contiguous nucleotides of the antiproliferative microRNA.

In certain embodiments, the antagomir is at least nineteen nucleotides in length.

In certain embodiments, the antagomir is stabilized against nucleolytic degradation.

In certain embodiments, the antagomir comprises a phosphorothioate backbone modification.

In certain embodiments, the phosphorothioate modification is at least at the first two internucleotide linkage at the 5′ end of the nucleotide sequence.

In certain embodiments, the phosphorothioate modification is at least at the first four internucleotide linkage at the 3′ end of the nucleotide sequence.

In certain embodiments, the phosphorothioate modification is at the first two internucleotide linkage at the 5′ end of the nucleotide sequence, and at the first four internucleotide linkage at the 3′ end of the nucleotide sequence.

In certain embodiments, the antagomir further comprises a 2′-modified nucleotide.

In certain embodiments, the 2′-modified nucleotide comprises a modification selected from the group consisting of: 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O—NMA).

In certain embodiments, the 2′-modified nucleotide comprises a 2′-O-methyl.

In certain embodiments, the antagomir further comprises a cholesterol molecule attached to the 3′ end of the agent.

In certain embodiments, the stem cells are contacted with the antagomir while in cell culture.

In certain embodiments, the antagomir is administered to a patient.

Another aspect of the invention provides a pharmaceutical preparation suitable for administration to a mammal for inducing or maintaining stem cells in vivo, comprising (i) an antagomir nucleic acid that inhibits an antiproliferative microRNA, and (ii) a pharmaceutically acceptable solvent, excipient, buffer and/or salt.

It is also contemplated that all embodiments of the invention, including those specifically described for different aspects of the invention, can be combined with any other embodiments of the invention as appropriate.

Other features and advantages of the invention will be apparent from the following detailed description and claims.

### BRIEF DESCRIPTION OF THE DRAWINGS

FIG. 1 shows the characterization of ALDH as a marker for progenitor cells in Comma-Dβ cells. FIG. 1A is a FACS pseudo color dot plot showing ALDH activity and Sca-1 expression in Comma-Dβ cells. In the left panel, cells were incubated with ALDEFLOUR substrate and stained with Sca-1. In the right panel, cells were stained with ALDEFLOUR and co-stained with Sca-1 and incubated with DEAB, to establish background fluorescence. Shown are 100,000 events. FIG. 1B is a histogram showing the colony-forming capacity of 4 sorted populations based on ALDH activity and Sca-1 expression seeded at clonal density on irradiated NIH3T3 feeders. Data represents the mean of four independent experiments. FIG. 1C shows Giemsa staining of ALDH^{bright} Sca-1^{high} colonies grown on irradiated feeders for 6 days. Based on morphology myoepithelial (top), luminal (middle), and mixture (bottom) colonies were observed. FIG. 1D is a histogram showing the colony-forming capacity of 4 sorted populations embedded at clonal density in Matrigel (n=4). FIG. 1E is a FACS profile of Comma-Dβ cells treated with a 6 μM dose of MAF for 4 days. Cells incubated with ALDEFLOUR and stained with Sca-1 (left), DEAB control (right). FIG. 1F shows cell viability assay after a 24 hr treatment with various doses of MAF of Comma-Dβ cells (black) and MAF resistant cells (blue). Data represents the mean±SD (error bar) of 2 independent experiments done in triplicate.

FIG. 2 demonstrates that microRNAs are differentially expressed in self-renewing compartments. Specifically, FIGS. 2A and 2B are bubble-plots depicting the relative abundance and log 2 ratio of miRNAs in ALDH^{bright} Sca-1^{high} and MAF-resistant cells (FIG. 2A) or relative to Sca-1^{neg} cells (FIG. 2B). FIG. 2C shows stem-loop semi-quantitative (q)RT-PCR for the mature forms of selected differentially expressed miRNAs. Shown are relative expression levels AACT of each miRNA from sorted Sca-1^{high} and Sca-1^{neg} Comma-Dβ cells.

FIG. 3 shows that enforced expression of miR-93 and/or let-7c depletes the self-renewing compartment in Comma-Dβ cells. FIG. 3A shows ectopic expression of Wnt expands the ALDH^{bright} Sca-1^{high} compartment. FACS plot of Comma-Dβ overexpressing Wnt-1 co-stained with ALDEFLOUR and Sca-1. FIG. 3B shows cell viability assay after a 48 hr treatment with various doses of MAF of Comma-Dβ cells (blue) and Wnt-1 expressing cells (black). Data represents the mean±SD (error bar) of 2 independent experiments done in triplicate. FIG. 3C shows FACS profile of empty vector control Comma-Dβ cells co-stained with ALDEFLOUR and Sca-1 (right), DEAB control for empty vector cells (middle), Comma-Dβ cells ectopically expressing Let-7c (right) and also stained with ALDEFLOUR and Sca-1. FIG. 3D shows FACS profile of empty vector control Comma-Dβ cells co-stained with ALDEFLOUR and Sca-1 (left 2 panels), Comma-Dβ cells ectopically expressing miR-93 (middle 2 panels), Comma-Dβ cells ectopically expressing Let-7c (right 2 panels). The top row shows data without the ALDH inhibitor DEAB, while the bottom row shows matching controls with the ALDH inhibitor DEAB. A depletion of the ALDH compartment is observed upon introduction of Let-7c or miR-93.

FIG. 4 shows self-renewal and differentiation of let-7c-negative cells in vitro. FIG. 4A is a cartoon depicting the let-7c sensor construct. FIG. 4B shows phase contrast images of Comma-Dβ cells expressing a construct with no let-7c binding sites (control) and Comma-Dβ cells expressing a sensor construct containing let-7c complementary sites. FIG. 4C is an overlay FACS dot plot of let-7c sensor cells (red) and uninfected Comma-Dβ cells (black) as an unstained control. DsR⁺ cells constitute 0.8% of the total population. FIG. 4D is a histogram showing the colony-forming ability of DsR⁺ and DsR⁻ cells embedded at clonal density in Matrigel (n=4). FIG. 4E is a phase contrast images of resultant DsR⁺ and DsR⁻ spheroids grown on Matrigel. DsR⁺ cells gave rise to substantially larger colonies (>50 μm) whereas DsR⁻ cells never exceeded this size. FIGS. 4F and 4G are confocal images of spheroids derived from DsR⁻ cells. FIG. 4F shows representative cross-section through the middle of a sphere co-stained with basal K5 and luminal K18 antibodies. FIG. 4G shows representative image through the top of a spheroid co-stained with basal K5 and α-Sma antibodies.

FIG. 5 is a FACS profile of Comma-Dβ cells stained with Hoescht 33342 Dye and with fluorescence displayed at two wavelength emissions, blue (FL7) and red (FL8), showing that Comma-Dβ cells contain a side-population (SP). FIG. 5A shows cells incubated in the absence of ATP transporter inhibitor, verapamil, and FIG. 5B shows cells stained and cultured in the presence of verapamil. As indicated by the FACS profile, SP represents approximately 2% of total number of events collected.

### DETAILED DESCRIPTION OF THE INVENTION

### 1. Overview

The instant invention is partly based on the discovery that aldehyde dehydrogenase (ALDH), together with Stem Cell Antigen (Sca-1), are mammary progenitor cell markers. By using ALDH or both markers, mammary progenitor cells can be isolated from cultured mammary cell lines harboring a permanent population of undifferentiated basal cells that are able to reconstitute the mammary tree, such as the Comma-Dβ cells. In a preferred embodiment, both markers are used to isolate the mammary progenitor cells to achieve much higher specificity compared with using either marker alone.

Thus one aspect of the invention provides a method for identifying, isolating, or enriching mammary progenitor cells, comprising isolating, from a population of candidate cells, cells that preferentially express aldehyde dehydrogenase (ALDH).

In a preferred embodiment, the method comprises isolating, from a population of candidate cells, cells that preferentially express aldehyde dehydrogenase (ALDH) and Stem Cell Antigen (Sca-1).

Since high ALDH activity is a hallmark for mammary progenitor cells, the invention also provides a method for identifying, isolating, or enriching mammary progenitor cells, comprising contacting a population of candidate cells (such as mammary epithelial cells known to harbor a subpopulation of mammary progenitor cells) with an agent that preferentially eliminates cells having low ALDH activity.

For example, ALDH activity helps to resist the killing effect of a class of anticancer drugs known as oxazaphosphorines. Mammary progenitor cells expressing high levels of ALDH resist the killing effect of oxazaphosphorines, or any agent that preferentially eliminates cells having low ALDH activity. Thus oxazaphosphorine may be used in the subject method for isolating mammary progenitor cells. A representative oxazaphosphorine is the chemotherapeutic drug mafosfamide (MAF).

The subject mammary progenitor cells are characterized by one or more of the following: (1) they are capable of reconstituting a functional mammary gland upon transplantation of a sufficient amount of the mammary progenitor cell into a host; (2) they are capable of self-renewal; (3) they are capable of differentiation into both myoepithelial and luminal cells in vitro and/or in vivo, and/or, (4) they are capable of population expansion and/or exhibiting increased mammosphere forming capacity upon enforced expression of β-catenin or Wnt-1.

These mammary progenitor cells may be isolated from a variety of sources, especially sources known to contain a population of mammary epithelial cells capable of self-renewal. For example, the starting population of candidate cells may be from a mammary epithelial cell line, such as the Comma-Dβ cell line.

There are many art recognized assays for characterizing one or more characteristics of the subject mammary progenitor cells, including (but are not limiting to): 2-D culture, 3-D culture, mammosphere assay, in vivo morphogenic potential assay, and colony formation assay.

In one embodiment, the subject mammary progenitor cells that preferentially express ALDH and/or Sca-1 are isolated by comparing ALDH activity in the presence or absence of an ALDH inhibitor.

ALDH activity can be measured in living cells by, for example, using a fluorogenic substrate, such as ALDEFLUOR (Corti et al., 2006, Hess et al., 2006, all incorporated by reference). ALDH induces retention of this substrate, resulting in increased florescence. Thus truly ALDH positive cells can be identified by comparison to cells cultured in ALDEFLUOR in the presence of an ALDH inhibitor, such as DEAB.

Thus in certain embodiments, preferential ALDH expression is manifested by having statistically significant ALDH activity in the absence of the ALDH inhibitor as compared to ALDH activity in the presence of ALDH inhibitor. Preferably, the ratio between the levels of the measured ALDH activity is at least about 10%, 30%, 50%, 100%, 2-fold, 5-fold, 10-fold, 50-fold, 100-fold, 200-fold, 300-fold, 500-fold or more compared to the control.

The method of the invention is highly sensitive and efficient. In certain embodiments, the subject mammary progenitor cells (e.g., cells that preferentially express ALDH and/or Sca-1) may constitute no more than 10%, 8%, 5%, 3%, 2%, 1% or less of the population of candidate cells.

In a related aspect, certain tumor initiating cells, such as those found in breast cancer, also exhibit high ALDH activity. Thus a related method of the invention concerns identifying, isolating, or enriching breast tumor-initiating cells or breast tumor stem cells (e.g., those cells that, when introduced in sufficient amount into a suitable host, can establish cancer in the host), comprising contacting a population of breast tumor cells with an agent that preferentially eliminates cells having low ALDH activity. These tumor-initiating cells or tumor stem cells can then be used in further research, such as cancer drug screening & development, or studying the property of tumor-initiating cells or tumor stem cells.

The instant invention is also partly based on the discovery that miRNA expression profiles may be used for characterization and/or isolation of certain cell populations or subpopulations, such as stem cell or progenitor cell populations. Preferred stem cell or progenitor cell is mammary progenitor cell or breast tumor-initiating cell/breast tumor stem cell.

microRNAs (miRNAs) are a class of evolutionary conserved, approximately 22-nucleotide non-coding RNAs that have recently emerged as important regulators of gene expression. They are involved in the regulation of many key biological processes by influencing the translational status of the transcriptome.

As used herein, “miRNA expression profile” or “miRNA signature” refers to the unique pattern of expression of a cell population or subpopulation, preferably a relatively homogeneous cell population or subpopulation (such as a stable cell line, or a stem cell line/progenitor cell line capable of self-renewal). The expression profile or signature is characterized by higher or lower expression levels of certain miRNA species, and/or the presence or absence of certain miRNA species, as compared to a proper control.

Because of the somewhat unique miRNA expression profile of the cell population or subpopulation, they can be identified, isolated, or enriched from a larger population of cells that do not necessarily share the same miRNA expression profile.

Although unique miRNA expression profiles have been associated with certain cell types, such as cancer cells, it wasn't clear prior to the instant invention that certain stem cell/progenitor cells, especially mammary progenitor cells, possess somewhat unique miRNA expression profiles. The ability to isolate mammary progenitor cells, for example, by using a combination of the mammary progenitor cell markers, such as ALDH and/or Sca-1, allows Applicants to identify unique miRNA expression profiles of the mammary progenitor cells. In turn, the miRNA expression profile may also be used to identify, isolate, or enrich mammary progenitor cells from a larger population of candidate cells known to possess such progenitor cells.

Thus another aspect of the invention provides a method for determining a microRNA (miRNA) expression profile of a population of progenitor cells, such as mammary progenitor cells, the method comprising: (1) obtaining the population of (mammary) progenitor cells and a control population of cells; (2) obtaining miRNA expression profiles for the population of (mammary) progenitor cells and the control population of cells; (3) comparing the miRNA expression profile for the population of (mammary) progenitor cells with that of the control population of cells, and, (4) identifying one or more miRNA that is expressed at a statistically significantly higher or lower level in the population of (mammary) progenitor cells compared to the control population of cells, thereby determining the miRNA expression profile of the population of (mammary) progenitor cells.

In certain embodiments, the population of mammary progenitor cells is progenitor cells from normal/healthy tissue.

In certain embodiments, the population of mammary progenitor cells is progenitor cells from pre-cancerous tissues or tumor tissues.

Characteristics of the mammary progenitor cells are described above. For example, the subject mammary progenitor cells preferentially express ALDH and/or Sca-1, and may be obtained by any of the methods described herein.

miRNA expression profiles may be determined by any art recognized methods. In certain embodiments, the miRNA expression profiles are determined by using miRNA microarray, deep sequencing analysis, and/or quantitative stem-loop PCR (qRT-PCR).

In certain embodiments, the control population of cells expresses low level of Sca-1 or no detectable level of Sca-1.

In certain embodiments, the population of mammary progenitor cells is tumor progenitor cells, and the control population of cells is matched healthy cells.

Such tumor progenitor cells are useful for many research or drug development projects. Thus one aspect of the invention provides a method of screening for a drug useful for cancer treatment, comprising: (1) contacting the subject tumor progenitor cells with a candidate compound; (2) determining whether the candidate compound inhibits proliferation and/or survival, or promotes benign differentiation of the tumor progenitor cells; wherein an observed inhibition of proliferation and/or survival, and/or enhanced benign differentiation of the tumor progenitor cells is indicative that the candidate compound is potentially useful as the drug for cancer treatment.

The invention also relates to an isolated mammary progenitor cell that preferentially expresses ALDH and/or Sca-1.

The invention also relates to an isolated mammary progenitor cell that preferentially expresses miR-205 and/or miR-22.

The invention also relates to an isolated mammary progenitor cell that substantially lacks expression of let-7b, let-7c, and/or miR-93.

As used herein “preferentially express” refers to a statistically significant higher expression level than a proper control.

For example, ALDH activity can be measured in living cells by using a fluorogenic substrate, ALDEFLUOR (Corti et al., 2006, Hess et al., 2006). ALDH induces retention of this substrate, resulting in increased florescence. Truly positive cells can be identified by comparison to cells cultured in ALDEFLUOR in the presence of an ALDH inhibitor, such as DEAB. Thus in certain embodiments, preferential ALDH expression is manifested by have statistically significant ALDH activity in the absence of the ALDH inhibitor as compared to ALDH activity in the presence of ALDH inhibitor. Preferably, the ratio between the levels of the measured ALDH activity is at least about 10%, 30%, 50%, 100%, 2-fold, 5-fold, 10-fold, 50-fold, 100-fold, 200-fold, 300-fold, 500-fold or more compared to the control.

Similarly, the cut-off value for Sca-1-positive and -negative cells can be determined.

Preferably, the ratio between the levels of the measured respective miRNA is at least about 10%, 20%, 30%, 50%, 75%, 100%, 2-fold, 3-fold, 4-fold, 5-fold, 10-fold more (or less in the case of decreased expression of certain miRNA in the mammary progenitor cell) compared to the control.

Applicants have identified, based on miRNA profiling, the characteristic expression pattern of miRNA for the subject mammary progenitor cells. Thus the invention also provides a method of identifying, isolating, or enriching mammary progenitor cells, comprising isolating, from a population of candidate cells, cells that preferentially express miR-205 and/or miR-22, or cells that substantially lack expression of let-7b, let-7c, and/or miR-93.

In certain embodiments, the cells that preferentially express miR-205 and/or miR-22 may be isolated by using one or more miRNA sensor, such as one described herein.

As used herein, “miRNA sensor” refers to molecules or constructs that may be used to detect the presence or absence of certain target miRNA, preferably in living cells. They offer a means to trace the expression of miRNA live, often without damaging the proliferation and/or differentiation of the cells expressing the miRNA and having the miRNA sensor. Exemplary embodiments of the miRNA sensor of the invention are described in more details below.

Thus in one aspect, the invention provides a method of identifying, isolating, or enriching mammary progenitor cells by tracing the expression of miR-205 and/or miR-22, which are preferentially expressed in the subject mammary progenitor cells, the method comprising: (1) introducing into a population of candidate cells an miRNA sensor that detects the presence of miR-205 and/or miR-22, wherein the presence of miR-205 and/or miR-22 eliminates the expression of a marker on the sensor; and, (2) identifying, isolating, or enriching cells that do not express the marker.

Since enforced expression of the preferentially expressed miRNA (such as miR-205 and/or miR-22) can expand the population of mammary progenitor cells within the starting population of candidate cells, a preferred embodiment of the method further comprise enforcing expression of miR-205 and/or miR-22 in the population of candidate cells before identifying, isolating, or enriching cells that do not express the marker. For example, miR-205 and/or miR-22 expression constructs may be introduced into the population of candidate cells before, after, or simultaneous with the sensor.

In a related embodiment, cells that substantially lack expression of let-7b, let-7c, and/or miR-93 are isolated by: (1) introducing into the population of candidate cells an miRNA sensor that detects the presence of let-7b, let-7c, and/or miR-93 by eliminating the expression of a marker; and, (2) isolating cells that express the marker.

Numerous miRNA sensors are suitable for the subject methods (see below). In certain embodiments, the miRNA sensor comprises: (1) a first polynucleotide sequence complementary to the sequence of one or more preferentially expressed miRNAs, such as miR-205 and/or miR-22, or the sequence of one or more miRNAs whose expression is substantially lacking, such as miRNA let-7b, let-7c, and/or miR-93; (2) a second polynucleotide sequence encoding a marker; wherein the presence of the miRNA (e.g., miR-205, miR-22, let-7b, let-7c, and/or miR-93) inhibits the expression of the marker.

In certain embodiments, the first polynucleotide and the second polynucleotide form a transcription unit, and the transcription product of the transcription unit is targeted for destruction by an RNAi mechanism in the presence of the miRNA (e.g., miR-205, miR-22, let-7b, let-7c, and/or miR-93).

In certain embodiments, the marker encodes an enzyme or a fluorescent protein. Suitable enzymes include (without limitation) alkaline phosphatases, beta-galactosidase, certain drug (puromycin, neomycin, hygromycin, etc.) resistance gene products, etc. Fluorescent proteins include (without limitation) DsRed or GFP, or a mutant thereof with a shifted emission maximum (YFP, BFP, EGFP, etc.). In certain embodiments, the marker may also be a toxin that may kill the cell that expresses the toxin.

In a related aspect, the invention also provides an miRNA sensor for sensing the presence of a target miRNA, comprising: (1) a first polynucleotide sequence complementary to the sequence of the target miRNA; (2) a second polynucleotide sequence encoding a fluorescent marker or a toxin marker; wherein the presence of the target miRNA inhibits the expression of the fluorescent marker or the toxin marker.

In certain embodiments, the first polynucleotide and the second polynucleotide form a transcription unit, and the transcription product of the transcription unit is targeted for destruction by an RNAi mechanism in the presence of the target miRNA.

In certain embodiments, the fluorescent marker is DsRed or GFP, or a mutant thereof with a shifted emission maximum.

In certain embodiments, the first polynucleotide is partially complementary (at least about 60%, 70%, 80%, 90%, 95%, 97%, 99% identical) to the sequence of the target miRNA. Preferably, the first polynucleotide can hybridize under high stringency conditions (as defined by standard molecular biology protocol, such as Sambrook et al., 1986) to the sequence of the target miRNA. In certain embodiments, the first polynucleotide perfectly matches the sequence of the target miRNA.

The subject sensor may have the capability to sense the presence or absence of multiple miRNA. Thus in certain embodiments, the first polynucleotide is further complementary to the sequence of a second target miRNA. Alternatively, multiple single-sensing sensors may be used together to achieve substantially the same result.

In certain embodiments, the first polynucleotide is located at the 3′-UTR region of the second polynucleotide sequence encoding the fluorescent marker or the toxin marker.

The instant invention also provide certain generic methods for cell identification, isolation, purification, or enrichment, based on the miRNA expression profiles of the target cell, and by using one or more sensors for the signature miRNA.

Thus in one aspect, the invention provides a method of identifying or isolating, from a population of candidate cells, a subpopulation of cells that preferentially express a target miRNA, the method comprising: (1) introducing into the population of candidate cells an miRNA sensor that detects the presence of the target miRNA by eliminating the expression of a marker; and, (2) isolating cells that do not express the marker.

In a related aspect, the invention provides a method of identifying or isolating, from a population of candidate cells, a subpopulation of cells that substantially lack expression of a target miRNA, the method comprising: (1) introducing into the population of candidate cells an miRNA sensor that detects the presence of the target miRNA by eliminating the expression of a marker; and, (2) isolating cells that express the marker.

In certain embodiments, the methods of the invention can be used to isolate a subpopulation of cells comprising no more than 10%, 8%, 5%, 3%, 2%, 1%, 0.5% or less of the population of candidate cells.

In certain embodiments, target subpopulation of cells are enriched at least about 200-fold, 100-fold, 50-fold, 35-fold, 20-fold, 10-fold from the population of candidate cells.

In certain embodiments, a combination of miRNAs and/or multiple sensors may be used. For example, the subject method may further comprises introducing into the population of candidate cells a second miRNA sensor that detects the presence of a second target miRNA by eliminating the expression of a second marker.

The second marker may be the same as the (first) marker. Alternatively, the second marker is different from the (first) marker. Different markers may be used in conjunction with one another (either concurrently or sequentially), or used separately (independent of one another).

Similarly, in a related aspect, the invention provides a method of deleting, from a population of candidate cells, a subpopulation of cells that preferentially express a target miRNA, the method comprising: (1) introducing into the population of candidate cells an miRNA sensor that detects the presence of the target miRNA by eliminating the expression of a marker; and, (2) eliminating/deleting cells that do not express the marker.

In a related aspect, the invention provides a method of deleting, from a population of candidate cells, a subpopulation of cells that substantially lack expression of a target miRNA, the method comprising: (1) introducing into the population of candidate cells an miRNA sensor that detects the presence of the target miRNA by eliminating the expression of a marker; and, (2) eliminating/deleting cells that express the marker.

In certain embodiments, the subpopulation of cells is tumor progenitor cells.

In certain embodiments, the marker is a toxin, and wherein the subpopulation of cells is tumor progenitor cells that lack the expression of the target miRNA. This allows one to selectively eliminate tumor progenitor cells based on their characteristic miRNA expression pattern that is not shared by normal cells, thereby increasing therapeutic index in cancer therapy.

Another aspect of the invention provides a method for expanding a subpopulation of mammary progenitor cells in a population of mammary epithelial cells comprising said mammary progenitor cells, the method comprising enforcing expression of miR-205 and/or miR-22, and/or inhibiting expression of let-7b, let-7c, and/or miR-93.

In certain embodiments, the expression of let-7b, let-7c, and/or miR-93 is inhibited by an antagomir that competitively inhibits RISC by binding to let-7b, let-7c, and/or miR-93, respectively.

In certain embodiments, the expression of let-7b, let-7c, and/or miR-93 is inhibited by inhibiting transcriptional or post-transcriptional processing of a precursor molecule for let-7b, let-7c, and/or miR-93, respectively.

In certain embodiments, the mammary epithelial cells are Comma-Dβ cells.

Another aspect of the invention provides a method for dedifferentiating a differentiated cell, comprising inhibiting the expression of let-7b, let-7c, and/or miR-93 in the differentiated cell.

In certain embodiments, the differentiated cell is reverted back to exhibit at least one progenitor/stem cell phenotype after the expression of let-7b, let-7c, and/or miR-93 is inhibited.

Those skilled in the art will recognize from the results disclose herein that antagomirs, i.e., antagonists of miRNA function, can be used to influence the cell fate.

Thus one aspect of the invention provides a method for regulating the state of differentiation of a normal, untransformed cell, comprising introducing an antagomir nucleic acid into the cell, which antagomir inhibits a microRNA that regulates one or more of differentiation or proliferation of the cell.

Another aspect of the invention provides a method for inducing dedifferentiation, comprising contacting a differentiated cell with an antagomir nucleic acid that inhibits an antiproliferative microRNA.

Yet another aspect of the invention provides a method for maintaining pluripotency of a stem cell, comprising contacting the stem cell with an antagomir nucleic acid that inhibits an antiproliferative microRNA.

In a related aspect, the invention also provides a pharmaceutical preparation suitable for administration to a mammal for inducing or maintaining stem cells in vivo, comprising (i) an antagomir nucleic acid that inhibits an antiproliferative microRNA, and (ii) a pharmaceutically acceptable solvent, excipient, buffer and/or salt.

The general feature of the invention having been described, the following section provides certain illustrative aspects of the invention that may be combined in specific embodiments described above. Other similar or equivalent art-recognized methods may also be readily adapted for use in the instant invention.

### II. MicroRNA Profiling

There are a number of art-recognized miRNA profiling methods, each can be used or adapted to be used with the subject invention. For example, miRNA profiling may be carried out by miRNA microarray, deep sequencing analysis, and/or quantitative stem-loop PCR (qRT-PCR), just to name a few.

In certain embodiments, small RNA library may be constructed from the cell line or any cell population (e.g., isolated cell population). The small RNA library is then subject to deep sequencing using, for example, the Illumina 1 G Genome Analyzer (for high throughput sequencing). The obtained sequences are then mapped to the suitable host genome, such as the mouse or human genome, using sequence alignment tools.

One exemplary sequence alignment tool is BLAT (Kent, BLAT—The BLAST-Like Alignment Tool. Genome Research 4: 656-664, 2002, incorporated by reference). BLAT is an alignment tool like NCBI's BLAST program (another suitable sequence alignment tool), but it is structured differently.

On DNA, BLAT works by keeping an index of an entire genome in memory. Thus, the target database of BLAT is not a set of GenBank sequences, but instead an index derived from the assembly of the entire genome. The index—which usually uses less than a gigabyte of RAM—consists of all non-overlapping 1′-mers except for those heavily involved in repeats. This smaller size allows BLAT to be far more easily mirrored. BLAT of DNA is designed to quickly find sequences of 95% and greater similarity of length 40 bases or more.

On proteins, BLAT uses 4-mers rather than 11-mers, finding protein sequences of 80% and greater similarity to the query of length 20+ amino acids. The protein index requires slightly more than 2 gigabytes of RAM. In practice, due to sequence divergence rates over evolutionary time, DNA BLAT works well within humans and primates, while protein BLAT continues to find good matches within terrestrial vertebrates and even earlier organisms for conserved proteins. Within humans, protein BLAT gives a much better picture of gene families (paralogs) than DNA BLAT. However, BLAST and psi-BLAST at NCBI can find much more remote matches.

From a practical standpoint, Blat has several advantages over BLAST: speed (no queues, response in seconds) at the price of lesser homology depth; the ability to submit a long list of simultaneous queries in FASTA format; five convenient output sort options; and alignment block details in natural genomic order.

BLAT is commonly used to look up the location of a sequence in the genome or determine the exon structure of an mRNA, but expert users can run large batch jobs and make internal parameter sensitivity changes by installing command line Blat on their own Linux server.

Sequence information obtained from the small RNA library may be mapped to existing database using BLAT. Suitable database for this purpose include the miRbase (Griffiths-Jones et al., miRBase: microRNA sequences, targets and gene nomenclature. Nucleic Acid Research, vol. 34, Database Issue, D140-D144, 2006, incorporated by reference), mouse non-coding RNA from NONCODE, which is an integrated knowledge database of non-coding RNAs from mouse (Liu et al, Nucleic Acids Research Vol. 33, Database issue D112-D115, 2005, incorporated by reference), tRNAs in “The RNA Modification Database” (Limbach et al., Summary: the modified nucleosides of RNA. Nucleic Acids Res. 22: 2183-2196, 1994, incorporated by reference), and rRNA entries in the Entrez Nucleotide Database.

In other embodiments, oligonucleotide microchips may be used to conduct genome-wide microRNA profiling. This type of studies has been done in human and mouse tissues. See Liu et al. (Proc. Natl. Acad. Sci. U.S.A. 101(26): 9740-9744, 2004), which describes miRNA gene expression profiling based on a microchip containing oligonucleotides corresponding to 245 miRNAs from human and mouse genomes. Using these microarrays, highly reproducible results were obtained that revealed tissue-specific miRNA expression signatures. The data were also confirmed by assessment of expression by Northern blots, real-time RT-PCR, and literature search. Such microchip oligolibrary can be expanded to include an increasing number of miRNAs discovered in various species and is useful for the analysis of normal and disease states.

miRNA profiling based on microchips may also be performed using commercially available services. For example, Exiqon (Woburn, Mass.) provides microRNA expression profiling service using its highly sensitive and specific miRCURY™ LNA Arrays. These arrays use LNA™ enhanced capture probes, which give greatly improved detection of miRNAs when compared with DNA-based arrays. This allows one to commit a minimum of sample to the miRNA profiling experiment. The highly sensitive LNA™ capture probes reportedly works on 1 μg total of RNA. The microRNA profiling service from Exiqon is available for all organisms.

Invitrogen's NCode™ miRNA Analysis product also provides sensitive, reproducible miRNA profiling.

Similarly, qRT-PCR may also be carried out using art recognized methods, or using commercially available services (see, for example, the Applied Biosystem's STEPONE™ and STEPONEPLUS™ Real-Time PCR Systems may be used for high performance real-time PCR).

### III. MicroRNA Sensors

The subject miRNA sensors are miRNA-sensitive sensor transgenes for detecting the presence and function of miRNA in cells. These miRNA sensor transgenes contained miRNA binding sites on reporter gene mRNAs, rendering expression of the reporter gene sensitive to the presence of the miRNA. One advantage of the miRNA sensors of the invention is that they can be used to sense the expression of miRNA in live cells and animals, often without the need to damage the live cells and animals. Thus the miRNA expression pattern may be determined in real time and in a dynamic fashion, thus greatly facilitating the studies focusing on the in vivo role of miRNAs.

Described herein are the exemplary embodiments of the subject miRNA sensor constructs, and related library that provides for spatial and temporal detection of miRNA expression and function in live cells and organisms. These miRNA constructs are suitable for real time and in situ detection of miRNA in these cells and organisms.

The subject miRNA sensor generally comprises: a first polynucleotide sequence complementary to a known or suspected miRNA sequence, an miRNA binding or target sequence, located in the 3′ UTR of an expression cassette capable of expression of a detectable marker or reporter protein. The marker or reporter protein may be a functional enzyme or protein (such as a toxin). The expression cassette can be delivered, optionally along with a control reporter gene, to a cell in vivo. If the miRNA is expressed and active in the cell, translation of the transcribed marker/reporter into the protein product is inhibited.

The marker/reporter protein is a protein that can be readily detected using methods known in the art, often without the need to sacrifice the animal, or perform an invasive procedure on the animal. For example, a preferred reporter protein is a fluorescent protein that can be traced in live cells by using a luminometer or similar devices in real time and to specific cells expression the marker, without sacrificing or harming the animal.

In one embodiment, the miRNA sensor construct contains a marker/reporter gene expression cassette that encodes a (fluorescent) reporter protein and contains transcription elements capable of (long term) expression of the reporter. An exemplary expression cassette is described in U.S. application Ser. No. 10/229,786, which is incorporated herein by reference. A preferred expression cassette comprises a suitable enhancer/promoter, such as the AFP (alpha-fetoprotein) enhancer and an albumin promoter. A preferred expression cassette further comprises a 5′ intron. Exemplary 5′ introns include, but are not limited to, the chimeric intron (from the pCI Mammalian Expression Vector, Promega, Madison, Wis.) and the human factor IX intron. A preferred expression cassette further comprises a 3′ UTR intron. An exemplary 3′ UTR intron is a truncated intron from the human albumin 3′ UTR. A preferred expression cassette further comprises one or more perfectly matched miRNA binding sites. The miRNA binding sites may also include binding sites that are not perfectly matched. The miRNA binding sites are preferably located in the 3′ UTR of the reporter gene expression cassette, but may also be located in other regions of the expression mRNA. To further reduce immunogenicity of the reporter construct, the construct can be optimized to reduce or eliminate CpG dinucleotides. The miRNA sensor plasmid may further comprise a second expression cassette that encodes a control reporter protein. Alternatively, a control reporter protein may be expressed from a gene on a separate construct and delivered together with the miRNA sensor construct.

In one embodiment, the miRNA sensor may be expressed long term. Long term expression of the reporter allows the investigator to monitor changes in miRNA expression or activity over time. Having a reporter protein that is fluorescent eliminates the need to sacrifice the cell, animal or tissue, therefore allowing the investigator to monitor miRNA expression of function over time in the same live cell, animal or tissue. These features permit one to determine if miRNAs are differentially active or expressed under different conditions, such as disease state, infection, fasting, response to changing environmental or developmental conditions, differential expression in different subpopulations of the cell line, etc.

The miRNA sensor can be delivered to cells in vitro using any art recognized methods, such as transfection, or to live organisms/tissues in vivo using gene delivery methods practiced in the art. Known gene delivery methods include: hydrodynamic intravascular delivery, including hydrodynamic tail vein injection, direct parenchymal injection, biolistic transfection, electroporation, lipid transfection (lipofection), polycation mediated transfection (polyfection), and lipid-polycation complex mediated transfection (lipopolyfection). A preferred delivery method (especially for murine) is hydrodynamic tail vein (HTV) injection. HTV injection provides a rapid, easy, reliable, nonsurgical method of polynucleotide delivery (U.S. Pat. No. 6,627,616, incorporated herein by reference). Another preferred delivery method is hydrodynamic limb vein (HLV) injection (U.S. patent application, incorporated herein by reference).

These subject miRNA sensors not only address the presence of miRNAs, but also the activity of these miRNAs.

More specifically, detection of miRNA activity is based on analysis of expression of a reporter gene that contains a miRNA binding site, preferable within the 3′ UTR of the reporter gene. If the cognate miRNA is expressed and functional in a cell, the miRNA will inhibit expression of the reporter gene. Inhibition of gene expression refers to a detectable decrease in the level of protein and/or mRNA product from a reporter/target gene. The level of inhibition of reporter gene activity can indicate the level of miRNA that is active in the cell. The reporter gene is expressed from a miRNA sensor plasmid which is delivered to cells in a desired tissue in an animal. The described miRNA sensor plasmids are capable of long term expression of a reporter gene if needed. By using a reporter protein that is fluorescent, it is possible to monitor miRNA at multiple time points in a single cell or animal. By using a sensor plasmid capable of long term expression of the reporter gene, the described miRNA sensor system allows an investigator to monitor changes in miRNA activity over time in the same cell/animal under a variety of treatment, environmental or developmental conditions.

The miRNA sensor plasmid comprises an expression cassette which a) encodes a marker or reporter protein, b) enables long term expression of the reporter gene and c) contains a miRNA binding site.

In one embodiment, the miRNA sensor plasmid that contains elements that allow for long-term expression of a transgene may be specifically expressed in selected tissues or developmental/differentiation stages, by, for example, using controllable promoters or enhancers. For example, tissue-specific, developmental stage specific, and/or inducible promoters may be used in conjunction with a minimal promoter to achieve these purposes.

A long-term enhancer/promoter combination is the albumin promoter together with the alpha-fetoprotein enhancer element. Other promoter/enhancer elements may be more appropriate for other long term expression in cell types in other tissues. Preferably, the described expression vector further comprises a 5′ intron and a 3′ intron. The 3′ UTR intron is located less than about 50 nucleotides downstream of the expression cassette translation stop codon. The 3′ intron is positioned to avoid non-sense mediated decay of the reporter gene mRNA.

The miRNA sensor plasmid contains a marker/reporter gene which encodes a marker/reporter protein (“marker” or “reporter” are used interchangeably herein). A reporter is a protein that can be quantitatively detected using methods known in the art. Typically, reporter proteins include enzymes, fluorescent proteins, and proteins or peptides that can be readily detected with antibodies. Enzymes are those proteins whose enzymatic activity can be measured. Reporter proteins commonly used in the art include both intracellular and secreted proteins. Examples include, but are not limited to: luciferase, β-galactosidase, chloramphenicol acetyl transferase, green fluorescent protein (and variants thereof), growth hormone, factor IX, secreted alkaline phosphatase, alpha 1-antitrypsin, and soluble CD4. For the present invention, fluorescent reporter genes are preferred.

An miRNA binding site is a nucleotide sequence which is complementary or partially complementary to at least a portion of a miRNA. The sequence can be a perfect match, meaning that the binding site sequence has perfect complementarity to the miRNA. Alternatively, the sequence can be partially complementary, meaning that one or more mismatches may occur when the miRNA is base paired to the binding site. Partially complementary binding sites preferably contain perfect or near perfect complementarity to the seed region of the miRNA. The seed region of the miRNA consists of the 5′ region of the miRNA from about nucleotide 2 to about nucleotide 8. For naturally occurring miRNAs and target genes, miRNAs with perfect complementarity to an mRNA sequence direct degradation of the mRNA through the RNA interference pathway while miRNAs with imperfect complementarity to the target mRNA direct translational control (inhibition) of the mRNA. The invention is not limited by which pathway is ultimately utilized by the miRNA in inhibiting expression of the reporter gene.

The miRNA binding site is preferably located in the 3′ untranslated region (UTR) of the reporter gene mRNA. In one embodiment, the miRNA binding site(s) are positioned just downstream of a 3′ UTR intron and about 100 nucleotides upstream of a polyadenylation signal. To facilitate cloning of a miRNA binding site into the miRNA sensor expression cassette, one or more restriction endonuclease sites are inserted into the 3′ UTR at the site of insertion of the miRNA binding site.

A control expression cassette encoding a second control reporter protein may be co-delivered with the miRNA sensor plasmid. The control reporter protein serves as an internal reference to normalize delivery efficiency of the miRNA sensor gene. A preferred control reporter protein may comprise a non-functional marker. The control expression cassette can be present on the same plasmid as the miRNA sensor gene, or it may be located on an independent plasmid which is co-delivered.

In one embodiment, an miRNA sensor plasmid library is formed. A miRNA sensor library comprises a set of miRNA sensor plasmids with independent and unique miRNA binding sites. A library may contain miRNA sensor plasmids for each of the known or suspected miRNAs in a species, in a specific tissue or cell type, or present at a specific developmental stage, or in a specific cell type (such as the subject mammary progenitor cells). In a preferred embodiment, the miRNA sensor library contains an exact match miRNA biding site for each desired miRNA. The availability of such a library will enable examination of expression of any number of known miRNA in the desired animal, tissue, or cell type. Lists of known miRNA sequences can be found in databases maintained by research organizations such as the Wellcome Trust Sanger Institute. The current number of known or suspected mouse miRNAs is more that 200 in miRBase release 7.1, and it is constantly being updated.

For delivery, any methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

The term “expression cassette” refers to a naturally, recombinantly, or synthetically produced nucleic acid molecule that is capable, of expressing a gene or genetic sequence in a cell. An expression cassette typically includes a promoter (allowing transcription initiation), and a sequence encoding one or more proteins or RNAs. Optionally, the expression cassette may include transcriptional enhancers, non-coding sequences, splicing signals and introns, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences. Optionally, the expression cassette may include a gene or partial gene sequence that is not translated into a protein.

The term gene generally refers to a nucleic acid sequence that comprises coding sequences necessary for the production of a nucleic acid (e.g., siRNA) or a polypeptide (protein) or protein precursor. A polypeptide can be encoded by a full length coding sequence or by any portion of the coding sequence so long as the desired activity or functional properties (e.g., enzymatic activity, ligand binding, signal transduction) of the full-length polypeptide or fragment are retained. In addition to the coding sequence, the term gene may also include, in proper contexts, the sequences located adjacent to the coding region on both the 5′ and 3′ ends which correspond to the full-length mRNA (the transcribed sequence) or all the sequences that make up the coding sequence, transcribed sequence and regulatory sequences. The sequences that are located 5′ of the coding region and which are present on the mRNA are referred to as 5′ untranslated region (5′ UTR). The sequences that are located 3′ or downstream of the coding region and which are present on the mRNA are referred to as 3′ untranslated region (3′ UTR). The term gene encompasses synthetic, recombinant, cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the mature mRNA transcript. Regulatory sequences include, but are not limited to, promoters, enhancers, transcription factor binding sites, polyadenylation signals, internal ribosome entry sites, silencers, insulating sequences, matrix attachment regions. Non-coding sequences may influence the level or rate of transcription and/or translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, nuclear transport, and immunogenicity. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

Long term expression means that the gene is expressed for greater than 2 weeks, greater than 4 weeks, greater than 8 weeks, greater than 20 weeks, greater than 30 weeks, or greater than 50 weeks with less than a 10-fold decrease in expression from day 1. Expression from typical CMV promoter driven gene expression cassettes typically drops by up to 1000-fold after 7 days. Expression for longer than a few weeks may require not eliciting an immune response to the expressed gene product, which is independent of the promoter/enhancer elements of the expression cassette. An immune response can be avoided or minimized by using immunosuppressive drugs, immune compromised animals, or expressing a gene product that is minimally or non-immunogenic.

The described miRNA sensor system can be used to study differences in miRNA activity in development, cellular differentiation, and metabolism. Currently, it is known that certain miRNAs are differentially expressed under different conditions or developmental/differentiation stages.

The long term expression miRNA sensor plasmids can be used to study differential expression and activity of these and other miRNAs is response to a variety of developmental and environmental conditions using a simple assay. The analysis of expression patterns of miRNAs can also provide clues as to their possible function and can be used to understand the function of miRNA in regulation of gene expression, including developmentally important gene or genes important in metabolism or disease.

The long term expression miRNA sensor plasmids can be used to investigate anti-miRNA molecules. MiRNA sensor plasmid can be used to evaluate the effectiveness of different types of miRNA inhibitors, including antisense miRNA oligonucleotides. The effectiveness of different oligonucleotide chemistries or modifications, in blocking miRNA activity, can be measured. Different oligonucleotide chemistries have been developed to enhance their activity. The miRNA sensor genes provide a rapid, reliable method to assess their effectiveness in vivo.

The use of anti-miRNA molecules targeting the endogenous miRNA of interest can provide a means to confirm results obtained from the miRNA sensor plasmid. If inhibition of the miRNA sensor gene is due to the presence of the cognate miRNA, co-delivery of the anti-miRNA molecule will result in relief of inhibition of reporter gene expression from the miRNA sensor plasmid. Antisense oligonucleotides complementary to endogenous miRNAs have been shown to transiently block miRNA function and therefore can be utilized and anti-miRNA molecules.

It is also possible to use an endogenous miRNA as a means of regulating expression of a transgene. By constructing a plasmid that encodes a gene of interest, instead of a reporter gene, and placing a specific miRNA binding site in the gene of interest, expression of the gene becomes sensitive to the miRNA phenotype of the cell-type to which the plasmid is delivered.

As an example, a plasmid can be constructed that codes for a toxic protein such as tumor necrosis factor-α(TNFα). A specific miRNA binding site can be placed in the 3′ UTR of the TNFα. If the plasmid is delivered to a cell that contains the cognate miRNA, the miRNA will inhibit expression of the TNFα gene in that cell. However, if the same plasmid is delivered to a cell that does not contain the cognate miRNA, TNFα is expressed, resulting in decreased viability of the cell. In this way, a cancer cell, especially a tumor progenitor/stem cell, or other desired cell, may be selectively targeted for expression of the transgene, by selecting a miRNA binding site that corresponds to a miRNA that is not expressed in the target cell, but is expressed in surrounding cells.

In a similar method, the process can be used to target expression of a transgene in cells that have a high level of a particular miRNA and while neighboring or non-target cells have little or none. For this process, a gene encoding a repressor or inhibitor of the transgene or encoded protein is co-delivered to the cell, preferably by encoding the repressor/inhibitor on the same plasmid as the transgene. By placing a miRNA binding site in the gene sequence of the repressor/inhibitor gene, expression of the repressor/inhibitor is dependent on the presence or absence of the cognate miRNA in the cell. If the plasmid is delivered to a cell of interest and the miRNA is present in the cell, the miRNA binds and causes inhibition of expression of the repressor/inhibitor mRNA. By reducing or eliminating expression of the repressor/inhibitor, expression or activity of the transgene is increased. Expression of the transgene in non-target cells is reduced because of the absence the miRNA, resulting in expression of the repressor/inhibitor and therefore repression or inhibition of the transgene.

As an example illustrating the process, a plasmid can be constructed that contains a TNFα repressor such as heat shock factor 1, in addition to the TNFα gene. An miRNA binding site is placed in the of the HSF-1 gene, wherein the miRNA is known to be expressed in the target cell, but not in non-target cells to which the plasmid may be delivered. If the plasmid is delivered to the desired targeted cells, the miRNA binds, expression of the repressor mRNA is inhibited and TNFα is expressed by the plasmid. If the plasmid is delivered to a non-target cells that lack the miRNA, the repressor/inhibitor is produced and TNFα is not expressed.

This targeting system could be used not only for eliminating harmful cells such as cancers, but used for targeting specific cells or tissues for expressing beneficial genes. When attempting to express a desired/beneficial gene, it may be desirable to only target a limited region so as not to over produce a large number of the gene product. The same process could be used to limit the target cells by including a specific miRNA-binding site in the plasmid to prevent the expression of the gene in non-target cells.

These plasmids could also be used in combination with existing antisense technology to produce a system in which expression can be regulated by delivering molecules to the cells that interfere with miRNA function or expression, such as antisense molecules. While these antisense molecules are intact, they prevent the production of a specific miRNA or inhibit binding of the miRNA to the miRNA-binding site in the gene of interest, which in turn allows for the expression of the gene of interest. After the antisense molecules are degraded or are removed, the miRNAs can then bind to the binding site on the plasmid and inhibit expression of the gene of interest.

The combination of the expression plasmid with delivery of an antisense molecule could also be used to form an inducible expression plasmid.

The term polynucleotide, or nucleic acid, is a term of art that refers to a polymer containing at least two nucleotides. Nucleotides are, the monomeric units of polynucleotide polymers. Polynucleotides with less than 120 monomeric units are often called oligonucleotides. Natural nucleic acids have a deoxyribose- or ribose-phosphate-backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, .beta.-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

The polynucleotide may contain sequences that do not serve a specific function in the target cell but are used in the generation of the polynucleotide. Such sequences include, but are not limited to, sequences required for replication or selection of the polynucleotide in a host.

A transfection reagent or delivery vehicle is a compound or compounds that bind(s) to or complex(es) with an inhibitor and mediates its entry into cells. Examples of transfection reagents include, but are not limited to, non-viral vectors, cationic liposomes and lipids, polyamines, calcium phosphate precipitates, histone proteins, polyethylenimine, and polylysine complexes. A non-viral vector is defined as a vector that is not assembled within an eukaryotic cell including protein and polymer complexes (polyplexes), lipids and liposomes (lipoplexes), combinations of polymers and lipids (lipopolyplexes), and multilayered and recharged particles. It has been shown that cationic proteins like histones and protamines, or synthetic polymers like polylysine, polyarginine, polyornithine, DEAE dextran, polybrene, and polyethylenimine may be effective intracellular delivery agents. Typically, the transfection reagent has a component with a net positive charge that binds to the oligonucleotide's or polynucleotide's negative charge. The transfection reagent mediates binding of oligonucleotides and polynucleotides to cells via its positive charge (that binds to the cell membrane's negative charge) or via ligands that bind to receptors in the cell. For example, cationic liposomes or polylysine complexes have net positive charges that enable them to bind to DNA or RNA.

A polynucleotide-based gene expression inhibitor comprises any polynucleotide containing a sequence whose presence or expression in a cell causes the degradation of or inhibits the function, transcription, or translation of a gene in a sequence-specific manner. Polynucleotide-based expression inhibitors may be selected from the group comprising: siRNA, microRNA, interfering RNA or RNAi, dsRNA, ribozymes, antisense polynucleotides, and DNA expression cassettes encoding siRNA, microRNA, dsRNA, ribozymes or antisense nucleic acids. RNAi molecules are polynucleotides or polynucleotide analogs that, when delivered to a cell, inhibit RNA function through RNA interference. Small RNAi molecules include RNA molecules less that about 50 nucleotides in length and include siRNA and miRNA. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding polynucleotides that direct destruction or translational repression of their mRNA targets. Antisense polynucleotides comprise sequence that is complimentary to a gene or mRNA. Antisense polynucleotides include, but are not limited to: morpholinos, DNA, RNA, 2′-O-methyl polynucleotides, and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA/gene is inhibited.

Antagonists of mRNA function or polynucleotide-based inhibitors can be used to influence cell fate. In one application, antagonists such as modified siRNAs or antagomirs are constructed using chemically-modified oligonucleotides. Modified siRNAs or antagomirs include molecules containing nucleotide analogues, including those molecules having additions, deletions, and/or substitutions in the nucleobase, sugar, or backbone; and molecules that are cross-linked or otherwise chemically modified. (See Crooke, U.S. Pat. Nos. 6,107,094 and 5,898,031; Elmen et al., U.S. Publication Nos. 2008/0249039 and 2007/0191294; Manoharan et al., U.S. Publication No. 2008/0213891; MacLachlan et al., U.S. Publication No. 2007/0135372; and Rana, U.S. Publication No. 2005/0020521; all of which are hereby incorporated by reference.)

### IV. Enforced microRNA Expression

miRNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Importantly, miRNAs are expressed in a highly tissue-specific or developmentally regulated manner and this regulation is likely key to their predicted roles in eukaryotic development and differentiation. Analysis of the normal role of miRNAs will be facilitated by techniques that allow the regulated over-expression or inappropriate expression of authentic miRNAs in vivo, whereas the ability to regulate the expression of siRNAs will greatly increase their utility both in cultured cells and in vivo. Thus one can design and express artificial microRNAs based on the features of existing microRNA genes, such as the gene encoding the human miR-30 microRNA. These miR30-based shRNAs have complex folds, and, compared with simpler stem/loop style shRNAs, are more potent at inhibiting gene expression in transient assays.

miRNAs are first transcribed as part of a long, largely single-stranded primary transcript (Lee et al., EMBO J. 21: 4663-4670, 2002). This primary miRNA transcript is generally, and possibly invariably, synthesized by RNA polymerase II (pol II) and therefore is normally polyadenylated and may be spliced. It contains an ˜80-nt hairpin structure that encodes the mature ˜22-nt miRNA as part of one arm of the stem. In animal cells, this primary transcript is cleaved by a nuclear RNaseIII-type enzyme called Drosha (Lee et al., Nature 425: 415-419, 2003) to liberate a hairpin miRNA precursor, or pre-miRNA, of ˜65 nt, which is then exported to the cytoplasm by exportin-5 and the GTP-bound form of the Ran cofactor (Yi et al., Genes Dev. 17: 3011-3016, 2003). Once in the cytoplasm, the pre-miRNA is further processed by Dicer, another RNaseIII enzyme, to produce a duplex of 22 bp that is structurally identical to an siRNA duplex (Hutvagner et al., Science 293: 834-838, 2001). The binding of protein components of the RNA-induced silencing complex (RISC), or RISC cofactors, to the duplex results in incorporation of the mature, single-stranded miRNA into a RISC or RISC-like protein complex, whereas the other strand of the duplex is degraded (Bartel, Cell 116: 281-297, 2004).

The miR-30 architecture can be used to express miRNAs or siRNAs from pol II promoter-based expression plasmids. See also Zeng et al, Methods in Enzymology 392: 371-380, 2005 (incorporated herein by reference). Also see the co-pending U.S. Ser. No. 11/444,107, filed on May 31, 2006 (incorporated herein by reference).

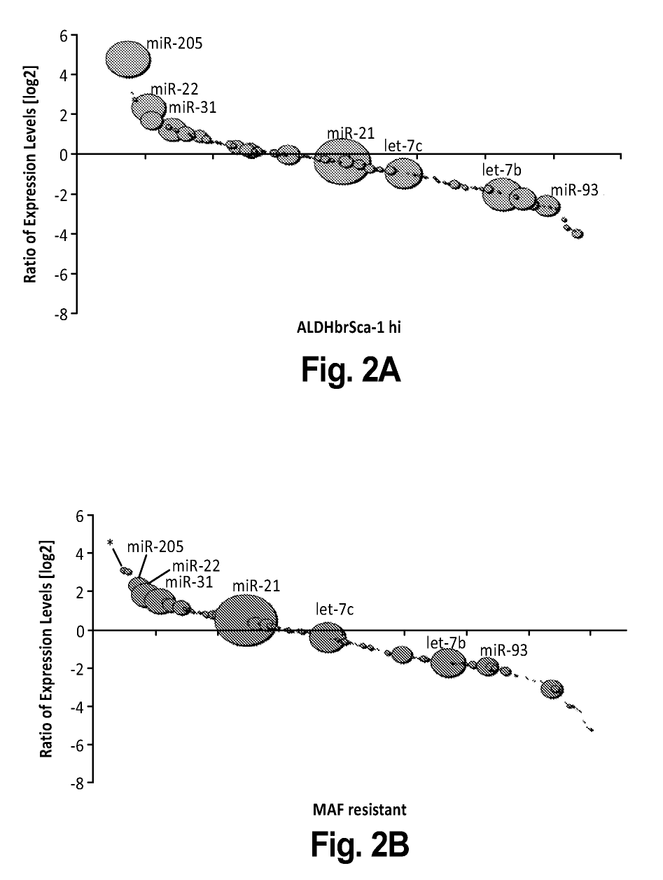

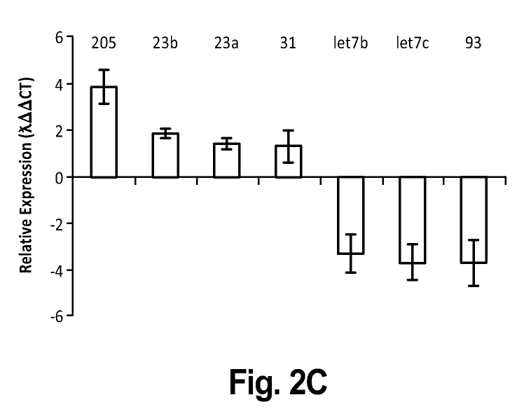

FIG. 2B of Zeng (supra) shows the predicted secondary structure of the miR-30 precursor hairpin (“the miR-30 cassette”). Boxed are extra nucleotides that were added originally for subcloning purposes (Zeng and Cullen, RNA 9: 112-123, 2003; Zeng et al., Mol. Cell. 9: 1327-1333, 2002). They represent XhoI-BglII sites at the 50 end and BamHI-XhoI sites at the 30 end. These appended nucleotides extend the minimal miR-30 precursor stem shown by several basepairs, similar to the in vivo situation where the primary miR-30 precursor is transcribed from its genomic locus (Lee et al., Nature 425: 415-419, 2003), and an extended stem of at least 5 bp is essential for efficient miR-30 production. Based on the numbering in FIG. 2B, mature miR-30 is encoded by nucleotides 44 to 65 and anti-miR-30 by nucleotides 3 to 25 of this precursor. In the simplest expression setting, the cytomegalovirus (CMV) immediate early enhancer/promoter may be used to transcribe the miR-30 cassette. The cassette is preceded by a leader sequence of approximately 100 nt and followed by approximately 170 nt before the polyadenylation site (Zeng et al., Mol. Cell. 9: 1327-1333, 2002). These lengths are arbitrary and can be longer or shorter. Mature 22-nt miR-30 can be made from such constructs.

Several other authentic miRNAs have been over-expressed by using analogous RNA pol II-based expression vectors or even pol III-dependent promoters (Chen et al., Science 303: 83-86, 2004; Zeng and Cullen, RNA 9: 112-123, 2003). Expression simply requires the insertion of the entire predicted miRNA precursor stem-loop structure into the expression vector at an arbitrary location. Because the actual extent of the precursor stem loop can sometimes be difficult to accurately predict, it is generally appropriate to include 50 bp of flanking sequence on each side of the predicted 80-nt miRNA stem-loop precursor to be sure that all cis-acting sequences necessary for accurate and efficient Drosha processing are included (Chen et al., Science 303: 83-86, 2004).

In an exemplary embodiment, to make the miR-30 expression cassette, the sequence from +1 to 65 (excluding the 15-nt terminal loop of the miR-30 cassette, FIG. 2B of Zeng) may be replaced as follows: the sequence from nucleotides 39 to 61, which is perfectly complementary to a target gene sequence, will act as the active strand during RNAi. The sequence from nucleotides 2 to 23 is thus designed to preserve the double-stranded stem in the miR-30-target cassette, but nucleotide +1 is now a C, to create a mismatch with nucleotide 61, a U, just like nucleotides 1 and 65 in the miR-30 cassette (FIG. 2B). Because the 30 arm of the stem (miR-30-target) is the active component for RNAi, changes in the 50 arm of the stem will not affect RNAi specificity. A 2-nt bulge may be present in the stem region of the authentic miR-30 precursor (FIG. 2B of Zeng). A break in the helical nature of the RNA stem may help ward off nonspecific effects, such as induction of an interferon response (Bridge et al., Nat. Genet. 34: 263-264, 2003) in expressing cells. This may be why miRNA precursors almost invariably contain bulges in the predicted stem. The miR-30 cassette in FIG. 2A of Zeng is then substituted with the miR-30-target cassette, and the resulting expression plasmid can be transfected into target cells.

The use of pol II promoters, especially when coupled with an inducible expression system (such as the TetOFF system of Clontech) offers flexibility in regulating the production of miRNAs in cultured cells or in vivo. Selection of stable cell lines leads to less leaky expression in the absence of the activator or presence of doxycycline, and therefore a stronger induction.

In certain embodiments, it would be advantageous if the antisense strand, for example, of the above miR-30-target construct is preferentially made as a mature miRNA, because its opposite strand does not have any known target. The relative basepairing stability at the 50 ends of an siRNA duplex is a strong determinant of which strand will be incorporated into RISC and hence be active in RNAi; the strand whose 50 end has a weaker hydrogen bonding pattern is preferentially incorporated into RISC, the RNAi effecter complex (Khvorova et al., Cell 115: 209-216, 2003; Schwarz et al., Cell 115: 208-299, 2003). This same principle can also be applied to the design of DNA vector-based siRNA expression strategies, including the one described here. However, for artificial miRNAs, the fact that the internal cleavage sites by Drosha and Dicer cannot be precisely predicted at present adds a degree of uncertainty as a 1- or 2-nt shift in the cleavage site can generate rather different hydrogen bonding patterns at the 50 ends of the resulting duplex, thus changing which strand of the duplex intermediate is incorporated into RISC. This is in contrast to the situation with synthetic siRNA duplexes, which have defined ends. On the other hand, any minor heterogeneity at the ends of an artificial miRNA duplex intermediate might not be a problem, as the miRNAs would still be perfectly complementary to their target.

The role of internal loop, stem length, and the surrounding sequences on the expression of miRNAs from miR-30-derived cassettes may also be systematically examined to optimize expression of the miR-based shRNA. Such analyses may suggest design elements that would maximize the yield of the intended RNA products. On the other hand, some heterogeneity could be inevitable. In addition to the 50-end rule, specific residues at some positions within an siRNA may also enhance siRNA function (Reynolds et al., Nat. Biotech. 22: 326-330, 2004).

In general, picking a target region with more than 50% AU content and designing a weak 50 end base pair on the antisense strand would be a good starting point in the design of any artificial miRNA/siRNA expression plasmid (Khvorova et al., Cell 115: 209-216, 2003; Reynolds et al., Nat. Biotech. 22: 326-330, 2004; Schwarz et al., Cell 115: 208-299, 2003).

In certain embodiments, expression of the miR-30 cassette may be in the antisense orientation, especially when the cassette is to be used in lentiviral or retroviral vectors. This is partly because miRNA processing may result in the degradation of the remainder of the primary miRNA transcript.

In other embodiments, vectors may contain inserts expressing more than one miRNAs. In such constructs, the fact that each miRNA stem-loop precursor is independently excised from the primary transcript by Drosha cleavage to give rise to a pre-miRNA allows simultaneous expression of several artificial or authentic miRNAs by a tandem array on a precursor RNA transcript.

In certain embodiments, the methods for efficient expression of microRNA involve the use of a precursor microRNA molecule having a microRNA sequence in the context of microRNA flanking sequences. The precursor microRNA is composed of any type of nucleic acid based molecule capable of accommodating the microRNA flanking sequences and the microRNA sequence. Examples of precursor microRNAs and the individual components of the precursor (flanking sequences and microRNA sequence) are provided herein.

In one aspect a precursor microRNA molecule is an isolated nucleic acid including microRNA flanking sequences and having a stem-loop structure with a microRNA sequence incorporated therein. An “isolated molecule” is a molecule that is free of other substances with which it is ordinarily found in nature or in vivo systems to an extent practical and appropriate for its intended use. In particular, the molecular species are sufficiently free from other biological constituents of host cells or if they are expressed in host cells they are free of the form or context in which they are ordinarily found in nature. For instance, a nucleic acid encoding a precursor microRNA having homologous microRNA sequences and flanking sequences may ordinarily be found in a host cell in the context of the host cell genomic DNA. An isolated nucleic acid encoding a microRNA precursor may be delivered to a host cell, but is not found in the same context of the host genomic DNA as the natural system. Alternatively, an isolated nucleic acid is removed from the host cell or present in a host cell that does not ordinarily have such a nucleic acid sequence. Because an isolated molecular species of the invention may be admixed with a pharmaceutically-acceptable carrier in a pharmaceutical preparation or delivered to a host cell, the molecular species may comprise only a small percentage by weight of the preparation or cell. The molecular species is nonetheless isolated in that it has been substantially separated from the substances with which it may be associated in living systems.

An “isolated precursor microRNA molecule” is one which is produced from a vector having a nucleic acid encoding the precursor microRNA. Thus, the precursor microRNA produced from the vector may be in a host cell or removed from a host cell. The isolated precursor microRNA may be found within a host cell that is capable of expressing the same precursor. It is nonetheless isolated in that it is produced from a vector and, thus, is present in the cell in a greater amount than would ordinarily be expressed in such a cell.

“MicroRNA flanking sequence” as used herein refers to nucleotide sequences including microRNA processing elements. MicroRNA processing elements are the minimal nucleic acid sequences which contribute to the production of mature microRNA from precursor microRNA. Often these elements are located within a 40 nucleotide sequence that flanks a microRNA stem-loop structure. In some instances the microRNA processing elements are found within a stretch of nucleotide sequences of between 5 and 4,000 nucleotides in length that flank a microRNA stem-loop structure.

Thus, in some embodiments the flanking sequences are 5-4,000 nucleotides in length. As a result, the length of the precursor molecule may be, in some instances at least about 150 nucleotides or 270 nucleotides in length. The total length of the precursor molecule, however, may be greater or less than these values. In other embodiments the minimal length of the microRNA flanking sequence is 10, 20, 30, 40, 50, 60, 70, 80, 90, 100, 150, 200 and any integer there between. In other embodiments the maximal length of the microRNA flanking sequence is 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900 4,000 and any integer there between.

The microRNA flanking sequences may be native microRNA flanking sequences or artificial microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Artificial microRNA flanking sequences are nucleotides sequences that are not found to be flanking to microRNA sequences in naturally existing systems. The artificial microRNA flanking sequences may be flanking sequences found naturally in the context of other microRNA sequences. Alternatively they may be composed of minimal microRNA processing elements which are found within naturally occurring flanking sequences and inserted into other random nucleic acid sequences that do not naturally occur as flanking sequences or only partially occur as natural flanking sequences.

The microRNA flanking sequences within the precursor microRNA molecule may flank one or both sides of the stem-loop structure encompassing the microRNA sequence. Thus, one end (i.e., 5′) of the stem-loop structure may be adjacent to a single flanking sequence and the other end (i.e., 3′) of the stem-loop structure may not be adjacent to a flanking sequence. Preferred structures have flanking sequences on both ends of the stem-loop structure. The flanking sequences may be directly adjacent to one or both ends of the stem-loop structure or may be connected to the stem-loop structure through a linker, additional nucleotides or other molecules.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches.

In some instances the precursor microRNA molecule may include more than one stem-loop structure. The multiple stem-loop structures may be linked to one another through a linker, such as, for example, a nucleic acid linker or by a microRNA flanking sequence or other molecule or some combination thereof.

In an alternative embodiment, useful interfering RNAs can be designed with a number of software programs, e.g., the OligoEngine siRNA design tool available at wwv.olioengine dot com. The siRNAs of this invention may range about, e.g., 19-29 basepairs in length for the double-stranded portion. In some embodiments, the siRNAs are hairpin RNAs having an about 19-29 bp stem and an about 4-34 nucleotide loop. Preferred siRNAs are highly specific for a region of the target gene and may comprise any about 19-29 bp fragment of a target gene mRNA that has at least one, preferably at least two or three, bp mismatch with a nontarget gene-related sequence. In some embodiments, the preferred siRNAs do not bind to RNAs having more than 3 mismatches with the target region.

### V. Expression Vectors and Host Cells

The invention also includes vectors for enforced expression of precursor microRNA molecules. Generally these vectors include a sequence encoding a precursor microRNA and (in vivo) expression elements. The expression elements include at least one promoter, such as a Pol II promoter, which may direct the expression of the operably linked microRNA precursor (e.g. the shRNA encoding sequence). The vector or primary transcript is first processed to produce the stem-loop precursor molecule. The stem-loop precursor is then processed to produce the mature microRNA.

RNA polymerase III (Pol III) transcription units normally encode the small nuclear RNA U6 (see Tran et al., BMC Biotechnology 3: 21, 2003, incorporate herein by reference), or the human RNAse P RNA Hi. However, RNA polymerase II (Pol II) transcription units (e.g., units containing a CMV promoter) are preferred for use with inducible expression. It will be appreciated that in the vectors of the invention, the subject shRNA encoding sequence may be operably linked to a variety of other promoters.

In some embodiments, the promoter is a type II tRNA promoter such as the tRNAVa promoter and the tRNAmet promoter. These promoters may also be modified to increase promoter activity. In addition, enhancers can be placed near the promoter to enhance promoter activity. Pol II enhancer may also be used for Pol III promoters. For example, an enhancer from the CMV promoter can be placed near the U6 promoter to enhance U6 promoter activity (Xia et al., Nuc Acids Res 31, 2003).

In certain embodiments, the subject Pol II promoters are inducible promoters. Exemplary inducible Pol II systems are available from Invitrogen, e.g., the GeneSwitch™ or T-REx™ systems; from Clontech (Palo Alto, Calif.), e.g., the TetON and TetOFF systems.

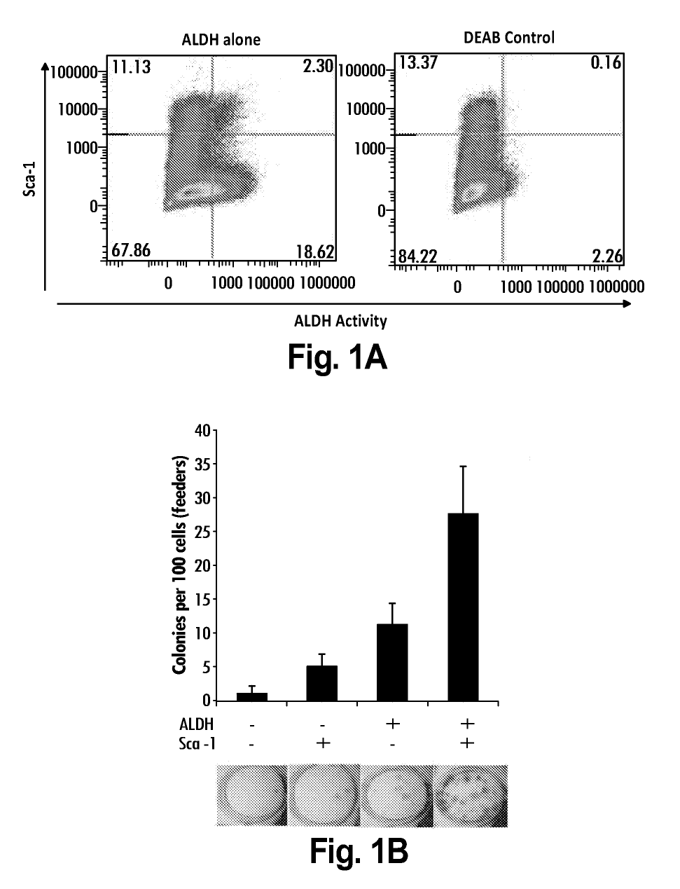

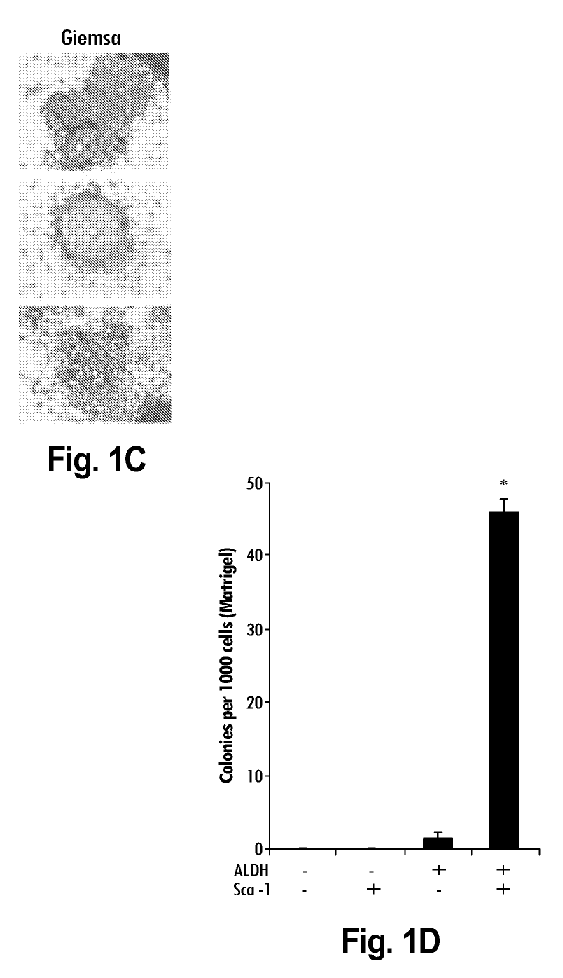

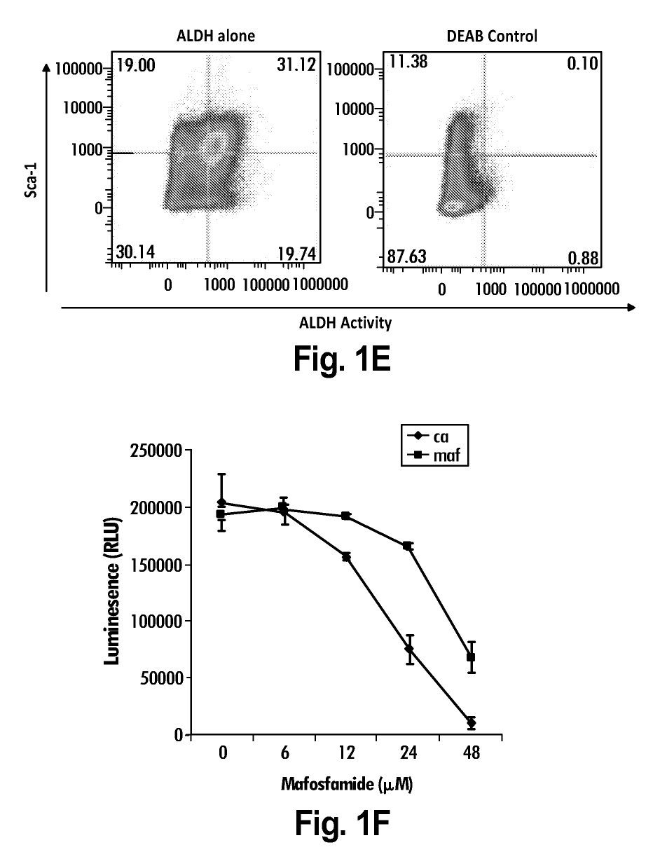

An exemplary Tet-responsive promoter is described in WO 04/056964A2 (incorporated herein by reference). See, for example, FIG. 1 of WO 04/056964A2. In one construct, a Tet operator sequence (TetOp) is inserted into the promoter region of the vector. TetOp is preferably inserted between the PSE and the transcription initiation site, upstream or downstream from the TATA box. In some embodiments, the TetOp is immediately adjacent to the TATA box. The expression of the subject shRNA encoding sequence is thus under the control of tetracycline (or its derivative doxycycline, or any other tetracycline analogue). Addition of tetracycline or Dox relieves repression of the promoter by a tetracycline repressor that the host cells are also engineered to express.

In the TetOFF system, a different tet transactivator protein is expressed in the tetOFF host cell. The difference is that Tet/Dox, when bind to an activator protein, is now required for transcriptional activation. Thus such host cells expressing the activator will only activate the transcription of an shRNA encoding sequence from a TetOFF promoter at the presence of Tet or Dox.

An alternative inducible promoter is a lac operator system, as illustrated in FIG. 2A of WO 04/056964 A2 (incorporated by reference). Briefly, a Lac operator sequence (LacO) is inserted into the promoter region. The LacO is preferably inserted between the PSE and the transcription initiation site, upstream or downstream of the TATA box. In some embodiments, the LacO is immediately adjacent to the TATA box. The expression of the RNAi molecule (shRNA encoding sequence) is thus under the control of IPTG (or any analogue thereof). Addition of IPTG relieves repression of the promoter by a Lac repressor (i.e., the LacI protein) that the host cells are also engineered to express. Since the Lac repressor is derived from bacteria, its coding sequence may be optionally modified to adapt to the codon usage by mammalian transcriptional systems and to prevent methylation. In some embodiments, the host cells comprise (i) a first expression construct containing a gene encoding a Lac repressor operably linked to a first promoter, such as any tissue or cell type specific promoter or any general promoter, and (ii) a second expression construct containing the dsRNA-coding sequence operably linked to a second promoter that is regulated by the Lac repressor and IPTG. Administration of IPTG results in expression of dsRNA in a manner dictated by the tissue specificity of the first promoter.

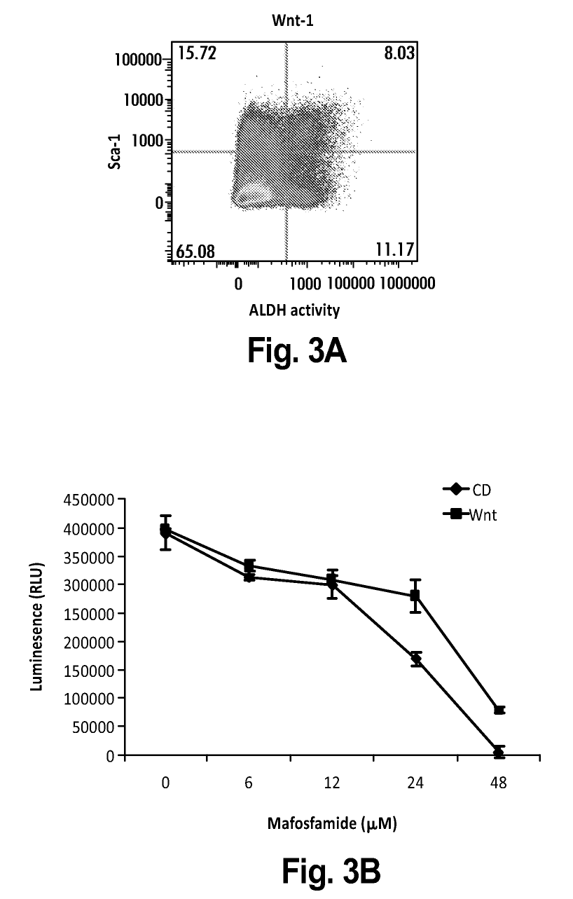

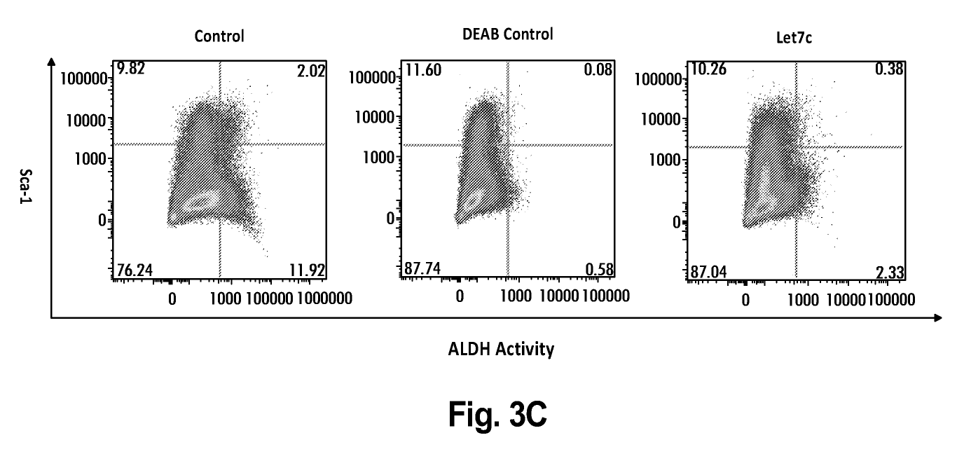

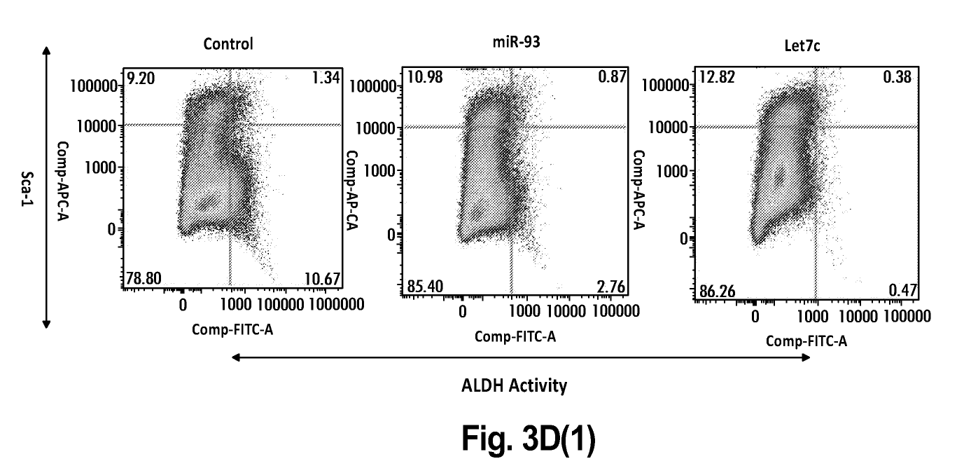

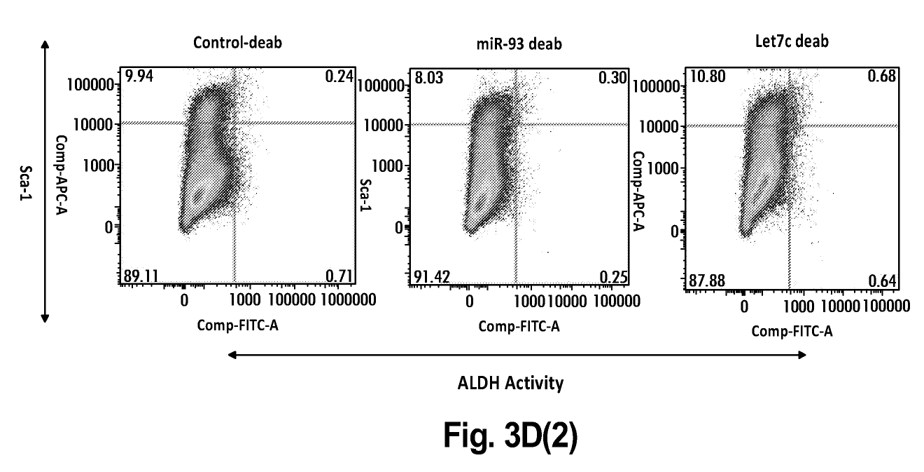

Yet another inducible system, a LoxP-stop-LoxP system, is illustrated in FIGS. 3A-3E of WO 04/056964 A2 (incorporated by reference). The RNAi vector of this system contains a LoxP-Stop-LoxP cassette before the hairpin or within the loop of the hairpin. Any suitable stop sequence for the promoter can be used in the cassette. One version of the LoxP Stop-LoxP system for Pol II is described in, e.g., Wagner et al., Nucleic Acids Research 25:4323-4330, 1997. The “Stop” sequences (such as the one described in Wagner, sierra, or a run of five or more T nucleotides) in the cassette prevent the RNA polymerase III from extending an RNA transcript beyond the cassette. Upon introduction of a Cre recombinase, however, the LoxP sites in the cassette recombine, removing the Stop sequences and leaving a single LoxP site. Removal of the Stop sequences allows transcription to proceed through the hairpin sequence, producing a transcript that can be efficiently processed into an open-ended, interfering dsRNA. Thus, expression of the RNAi molecule is induced by addition of Cre.

In some embodiments, the host cells contain a Cre-encoding transgene under the control of a constitutive, tissue-specific promoter. As a result, the interfering RNA can only be inducibly expressed in a tissue-specific manner dictated by that promoter. Tissue-specific promoters that can be used include, without limitation: a tyrosinase promoter or a TRP2 promoter in the case of melanoma cells and melanocytes; an MMTV or WAP promoter in the case of breast cells and/or cancers; a Villin or FABP promoter in the case of intestinal cells and/or cancers; a RIP promoter in the case of pancreatic beta cells; a Keratin promoter in the case of keratinocytes; a Probasin promoter in the case of prostatic epithelium; a Nestin or GFAP promoter in the case of CNS cells and/or cancers; a Tyrosine Hydroxylase, S100 promoter or neurofilament promoter in the case of neurons; the pancreas-specific promoter described in Edlund et al., Science 230: 912-916, 1985; a Clara cell secretory protein promoter in the case of lung cancer; and an Alpha myosin promoter in the case of cardiac cells.

Cre expression also can be controlled in a temporal manner, e.g., by using an inducible promoter, or a promoter that is temporally restricted during development such as Pax3 or Protein O (neural crest), Hoxal (floorplate and notochord), Hoxb6 (extraembryonic mesoderm, lateral plate and limb mesoderm and midbrain-hindbrain junction), Nestin (neuronal lineage), GFAP (astrocyte lineage), Lck (immature thymocytes). Temporal control also can be achieved by using an inducible form of Cre. For example, one can use a small molecule controllable Cre fusion, for example a fusion of the Cre protein and the estrogen receptor (ER) or with the progesterone receptor (PR). Tamoxifen or RU486 allow the Cre-ER or Cre-PR fusion, respectively, to enter the nucleus and recombine the LoxP sites, removing the LoxP Stop cassette. Mutated versions of either receptor may also be used. For example, a mutant Cre-PR fusion protein may bind RU486 but not progesterone. Other exemplary Cre fusions are a fusion of the Cre protein and the glucocorticoid receptor (GR). Natural GR ligands include corticosterone, cortisol, and aldosterone. Mutant versions of the GR receptor, which respond to, e.g., dexamethasone, triamcinolone acetonide, and/or RU38486, may also be fused to the Cre protein.

In certain embodiments, additional transcription units may be present 3′ to the shRNA portion. For example, an internal ribosomal entry site (IRES) may be positioned downstream of the shRNA insert, the transcription of which is under the control of a second promoter, such as the PGK promoter. The IRES sequence may be used to direct the expression of a operably linked second gene, such as a reporter gene (e.g., a fluorescent protein such as GFP, BFP, YFP, etc., an enzyme such as luciferase (Promega), etc.). The reporter gene may serve as an indication of infection/transfection, and the efficiency and/or amount of mRNA transcription of the shRNA—IRES—reporter cassette/insert. Optionally, one or more selectable markers (such as puromycin resistance gene, neomycin resistance gene, hygromycin resistance gene, zeocin resistance gene, etc.) may also be present on the same vector, and are under the transcriptional control of the second promoter. Such markers may be useful for selecting stable integration of the vector into a host cell genome, and may also be used as the marker of the subject miRNA sensor.

Certain exemplary vectors useful for expressing the precursor microRNAs are shown in the examples of the co-pending U.S. Ser. No. 11/444,107, filed on May 31, 2007 (incorporated by reference).

In general, variants typically will share at least 40% nucleotide identity with any of the described vectors, in some instances, will share at least 50% nucleotide identity; and in still other instances, will share at least 60% nucleotide identity. The preferred variants have at least 70% sequence homology. More preferably the preferred variants have at least 80% and, most preferably, at least 90% sequence homology to the described sequences.

Variants with high percentage sequence homology can be identified, for example, using stringent hybridization conditions.

The term “stringent conditions”, as used herein, refers to parameters with which the art is familiar. More specifically, stringent conditions, as used herein, refer to hybridization at 65° C. in hybridization buffer (3.5×SSC, 0.02% Ficoll, 0.02% polyvinyl pyrolidone, 0.02% bovine serum albumin, 2.5 mM NaH₂PO₄ (pH 7), 0.5% SDS, 2 mM EDTA). SSC is 0.15M sodium chloride/0.15M sodium citrate, pH 7; SDS is sodium dodecyl sulphate; and EDTA is ethylenediaminetetra-acetic acid. After hybridization, the membrane to which the DNA is transferred is washed at 2×SSC at room temperature and then at 0.1×SSC/0.1×SDS at 65° C. There are other conditions, reagents, and so forth which can be used, which result in a similar degree of stringency. Such variants may be further subject to functional testing such that variants that substantially preserve the desired/relevant function of the original vectors are selected/identified.

The “in vivo expression elements” are any regulatory nucleotide sequence, such as a promoter sequence or promoter-enhancer combination, which facilitates the efficient expression of the nucleic acid to produce the precursor microRNA. The in vivo expression element may, for example, be a mammalian or viral promoter, such as a constitutive or inducible promoter or a tissue specific promoter. Constitutive mammalian promoters include, but are not limited to, polymerase II promoters as well as the promoters for the following genes: hypoxanthine phosphoribosyl transferase (HPTR), adenosine deaminase, pyruvate kinase, and β-actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. The promoters useful as in vivo expression element of the invention also include inducible promoters. Inducible promoters are expressed in the presence of an inducing agent. For example, the metallothionein promoter is induced to promote transcription in the presence of certain metal ions. Other inducible promoters are known to those of ordinary skill in the art.

One useful inducible expression system that can be adapted for use in the instant invention is the Tet-responsive system, including both the TetON and TetOFF embodiments.

TetOn system is a commercially available inducible expression system from Clontech Inc. This is of particular interest because current siRNA expression systems utilize pol III promoters, which are difficult to adapt for inducible expression. The Clontech TetON system includes the pRev-TRE vector, which can be packaged into retrovirus and used to infect a Tet-On cell line expressing the reverse tetracycline-controlled transactivator (rtTA). Once introduced into the TetON host cell, the shRNA insert can then be inducibly expressed in response to varying concentrations of the tetracycline derivate doxycycline (Dox).

In general, the in vivo expression element shall include, as necessary, 5′ non-transcribing and 5′ non-translating sequences involved with the initiation of transcription. They optionally include enhancer sequences or upstream activator sequences as desired.

Vectors include, but are not limited to, plasmids, phagemids, viruses, other vehicles derived from viral or bacterial sources that have been manipulated by the insertion or incorporation of the nucleic acid sequences for producing the precursor microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; lentiviruses; and RNA viruses such as any retrovirus. One can readily employ other unnamed vectors known in the art.

Viral vectors are generally based on non-cytopathic eukaryotic viruses in which non-essential genes have been replaced with the nucleic acid sequence of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Genetically altered retroviral expression vectors have general utility for the high-efficiency transduction of nucleic acids in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, “Gene Transfer and Expression, A Laboratory Manual,” W.H. Freeman Co., New York (1990) and Murry, Ed. “Methods in Molecular Biology,” vol. 7, Humana Press, Inc., Cliffton, N.J. (1991).

Exemplary vectors are disclosed herein and in US 2005/0075492 A2 (incorporated herein by reference) and WO 04/056964 A2 (incorporated herein by reference).

The invention also encompasses host cells transfected with the subject vectors, especially host cell lines with stably integrated shRNA constructs. In certain embodiments, the subject host cell contains only a single copy of the integrated construct expressing the desired shRNA (optionally under the control of an inducible and/or tissue specific promoter). Host cells include for instance, cells and cell lines (such as those harboring the subject progenitor cells). Exemplary cells include: primary cells, isolated progenitor cells, or cancer (progenitor/stem) cells, etc.

### VI. Antagomirs

MicroRNAs are transcribed from endogenous DNA and form hairpin structures (called pre-microRNAs) that are processed by an enzyme to form mature microRNA duplexes that are about 21 to 23 nucleotides long. A protein complex called RNA-induced silencing complex (RISC) allows the antisense strand of the microRNA to couple with matching messenger RNA (mRNA) sequences at 3′ untranslated regions (the bulge in the microRNA denotes a region found in microRNAs that is not complementary to the mRNA). The binding of the microRNA to mRNA disrupts the translation or processing of the message, thereby disrupting the expression of the protein.

In a recent study, Krützfeldt and colleagues (Nature 438: 685-689, 2005) showed that miRNA function can transiently be antagonized by antagomirs—chemically modified oligonucleotides complementary to individual miRNAs. In that study, a cholesterol-linked single-stranded RNA, or antagomir complementary to miR-122 (a microRNA that is highly expressed in the liver), was injected into mice. This antagomir-122 caused the depletion of miR-122 and decreased plasma cholesterol levels. Thus, miR-122 may down-regulate a repressor of genes in the cholesterol biosynthetic pathway, and antagomir-122 may enhance the expression of the repressor, which in turn inhibits the transcription of cholesterol-synthesizing enzymes. In other words, antagomir-122 may counter a brake on the production of a transcriptional repressor protein.

Those skilled in the art will recognize from the results disclose herein that antagomirs, i.e., antagonists of miRNA function, can be used to influence the cell fate.

Thus one aspect of the invention provides a method for regulating the state of differentiation of a normal, untransformed cell, comprising introducing an antagomir nucleic acid into the cell, which antagomir inhibits a microRNA that regulates one or more of differentiation or proliferation of the cell.

Another aspect of the invention provides a method for inducing dedifferentiation, comprising contacting a differentiated cell with an antagomir nucleic acid that inhibits an antiproliferative microRNA.

Yet another aspect of the invention provides a method for maintaining pluripotency of a stem cell, comprising contacting the stem cell with an antagomir nucleic acid that inhibits an antiproliferative microRNA.

In certain embodiments, the invention provides for inducing dedifferentiation of cells and/or maintenance of stem cell pluripotency by introducing into cells one or more antagomirs of miRNA's that otherwise suppress genes involved in proliferation or mitosis (“antiproliferative miRNA”) or which suppress expression of genes that negatively regulate differetiation (differentiation inducing miRNAs). The let-7 miRNA's are examples of antiproliferative miRNA's, and have also been termed “antioncogenic miRNA.” Antagonism of let-7 miRNA, such as let-7c, can cause an increase in expression of the proliferative signal, ras, and induce dedifferentiation of an otherwise differentiated cell, or can prevent differentiation of a stem cell so as to maintain it's pluripotency. Other examples of antiproliferative miRNA that can be inhibited by antagomirs include miRNA that otherwise (i) inhibit expression of growth factors or mitogens; (ii) inhibit expression of receptor tyrosine kinases such as epidermal growth factor receptor (EGFR), platelet-derived growth factor receptor (PDGFR), vascular endothelial growth factor receptor (VEGFR) or HER2/neu; (iii) inhibit cytoplasmic tyrosine kinases such as Src-family, Syk-ZAP-70 family, and BTK family of tyrosine kinases; and (iv) inhibit transcription factors that otherwise promote proliferation, such as the c-myc.

Examples of differentiation-inducing miRNA are those that promote the expression or function of a mitotic inhibitory gene or tumor suppressor (or “antioncogene”), merely to illustrate. Examples of differentiation-inducing miRNA that can be inhibited by antagomirs include miRNA that otherwise (i) upregulate expression or activity of a restinoblastoma (Rb) protein; (ii) upregulate expression or activity of a p53 (Rb) protein; (ii) upregulate expression or activity of a p16 (ink4) protein. Likewise, antagomers that inhibit miRNA's that down-regulate tumor suppressors can be used induce differentiation of stem cells as part of a process of producing particular cells or tissues.

Other examplary antagomirs are provided in the art, such as Meister et al. (RNA 10: 544-550, 2004; Krutzfeld et al. (Nature 438: 685-689, 2005; Krutzfeld et al. (Nucleic Acids Res. 35: 2885-2892, 2007; Scherr et al. (Nucleic Acid Res. epublished doi:10.1093/nar/gkm971, 2007; and US Patent Publications 20050182005 and 20070213292. The teachings of these references are incorporated by reference herein.

In certain embodiments, the subject antagomirs comprise a sequence that is substantially complementary to 12 to 23 contiguous nucleotides of the target miRNA, such as the antiproliferative microRNA. In certain embodiments, the antagomir is at least nineteen nucleotides in length, for example, about 23 nucleotides, or about 25 nucleotides. The tendency for improved activity of certain 25-mer antagomir can be explained on the basis of improved thermodynamic binding affinity of the 25 mer, which should also have higher biostability from exonucleases for the core 23 mer.

Optimum number of phosphorothioate modifications and minimum length of antagomirs for the biological function in vivo can be readily determined using, for example, suitable biological assays or binding affinity assays for the specific antagomirs.

In certain embodiments, antagomir nucleic acids are transcribed from a vector introduced into the host cell/organism. For example, the antagomir nucleic acid may be ectopically contacted with the target/host cell, and is taken up thereby. In fact, antagomirs may be expressed in the host cell or organism using any art recognized means for nucleic acid expression, such as lentivirus-mediated (antagomir) expression.

In certain embodiments, the antagomirs are stabilized against nucleolytic degradation. For example, the antagomir may comprises a phosphorothioate backbone modification. The phosphorothioate modification can be present at least at the first two internucleotide linkage at the 5′ end of the nucleotide sequence. The phosphorothioate modification can be present at least at the first four internucleotide linkage at the 3′ end of the nucleotide sequence. The phosphorothioate modification can be at the first two internucleotide linkage at the 5′ end of the nucleotide sequence, and at the first four internucleotide linkage at the 3′ end of the nucleotide sequence.

The subject antagomir may further comprises a 2′-modified nucleotide, such as a modification selected from the group consisting of: 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O—NMA). Preferably, the 2′-modified nucleotide comprises a 2′-O-methyl.

In certain embodiments, the antagomir further comprises a cholesterol molecule attached to the 3′ end of the agent.

In certain embodiments, the antagomir is administered to a patient, such as a human patient, or a non-human animal patient.

In a related aspect, the invention also provides a pharmaceutical preparation suitable for administration to a mammal for inducing or maintaining stem cells in vivo, comprising (i) an antagomir nucleic acid that inhibits an antiproliferative microRNA, and (ii) a pharmaceutically acceptable solvent, excipient, buffer and/or salt.

The dosage of antagomir can be readily determined based on a nunber of patient specific factors commonly known in the art. In many embodiments, antagomirs efficiently silence miRNAs in most tissues after three injections at 80 mg/kg bodyweight (bw) on consecutive days (e.g., 2, 3, 4, 5, 10 days, etc.). Other dosages can be readily derived.

Antagomirs or pharmaceutical preparations comprising the antagomirs can be delivered, for example, by intravenous injection in a small volume (0.2 ml, 80 mg/kg, 3 consecutive days) and normal pressure.

### VII. Exemplary Methods of Using

In certain aspects, methods of the invention comprise contacting and introducing into a target cell with a subject vector capable of expressing a precursor microRNA as described herein, to regulate the expression of a target gene in the cell. The vector produces the microRNA transcript, which is then processed into precursor microRNA in the cell, which is then processed to produce the mature functional microRNA, which is capable of altering accumulation of at least one target protein in the target cell. Accumulation of the protein may be effected in a number of different ways. For instance the microRNA may directly or indirectly affect translation or may result in cleavage of the mRNA transcript or even effect stability of the protein being translated from the target mRNA. MicroRNA may function through a number of different mechanisms. The methods and products of the invention are not necessarily limited to any one mechanism. The method may be performed in vitro, e.g., for studying gene function, ex vivo or in vivo, e.g. for therapeutic purposes.

An “ex vivo” method as used herein is a method which involves isolation of a cell from a subject, manipulation of the cell outside of the body, and reimplantation of the manipulated cell into the subject. The ex vivo procedure may be used on autologous or heterologous cells, but is preferably used on autologous cells. In preferred embodiments, the ex vivo method is performed on cells that are isolated from bodily fluids such as peripheral blood or bone marrow, but may be isolated from any source of cells. When returned to the subject, the manipulated cell will be programmed for cell death or division, depending on the treatment to which it was exposed. Ex vivo manipulation of cells has been described in several references in the art, including Engleman, E. G., 1997, Cytotechnology, 25:1; Van Schooten, W., et al., 1997, Molecular Medicine Today, June, 255; Steinman, R. M., 1996, Experimental Hematology, 24, 849; and Gluckman, J. C., 1997, Cytokines, Cellular and Molecular Therapy, 3:187. The ex vivo activation of cells of the invention may be performed by routine ex vivo manipulation steps known in the art. In vivo methods are also well known in the art. The invention thus is useful for therapeutic purposes and also is useful for research purposes such as testing in animal or in vitro models of medical, physiological or metabolic pathways or conditions.

The ex vivo and in vivo methods are performed on a subject. A “subject” shall mean a human or non-human mammal, including but not limited to, a dog, cat, horse, cow, pig, sheep, goat, primate, rat, and mouse, etc.

In some instances the mature microRNA is expressed at a level sufficient to cause at least a 2-fold, or in some instances, a 10 fold reduction in accumulation of the target protein. The level of accumulation of a target protein may be assessed using routine methods known to those of skill in the art. For instance, protein may be isolated from a target cell and quantitated using Western blot analysis or other comparable methodologies, optionally in comparison to a control. Protein levels may also be assessed using reporter systems or fluorescently labeled antibodies. In other embodiments, the mature microRNA is expressed at a level sufficient to cause at least a 2, 5, 15, 20, 25, 30, 35, 40, 45, 50, 55, 60, 65, 70, 75, or 100 fold reduction in accumulation of the target protein. The “fold reduction” may be assessed using any parameter for assessing a quantitative value of protein expression. For instance, a quantitative value can be determined using a label i.e. fluorescent, radioactive linked to an antibody. The value is a relative value that is compared to a control or a known value.

Different microRNA sequences have different levels of expression of mature microRNA and thus have different effects on target mRNA and/or protein expression. For instance, in some cases a microRNA may be expressed at a high level and may be very efficient such that the accumulation of the target protein is completely or near completely blocked. In other instances the accumulation of the target protein may be only reduced slightly over the level that would ordinarily be expressed in that cell at that time under those conditions in the absence of the mature microRNA. Complete inhibition of the accumulation of the target protein is not essential, for example, for therapeutic purposes. In many cases partial or low inhibition of accumulation may produce a preferred phenotype. The actual amount that is useful will depend on the particular cell type, the stage of differentiation, conditions to which the cell is exposed, the modulation of other target proteins, etc.

The microRNAs may be used to knock down gene expression in vertebrate cells for gene-function studies, including target-validation studies during the development of new pharmaceuticals, as well as the development of human disease models and therapies, and ultimately, human gene therapies.

In one aspect, the invention provides a method for dedifferentiating a differentiated cell, comprising inhibiting the expression of let-7b, let-7c, and/or miR-93 in the differentiated cell.

In certain embodiments, the differentiated cell is reverted back to exhibit at least one progenitor/stem cell phenotype after the expression of let-7b, let-7c, and/or miR-93 is inhibited.

The methods of the invention are also useful for treating any type of “disease”, “disorder” or “condition” in which it is desirable to increase or reduce the expression or accumulation of a particular target protein(s) and/or miRNA. For example, miRNA expression profiles of certain diseases, such as cancers, may be determined using the subject methods. The disease may be treated by overexpressing one or more miRNA known to be consistently lacking the diseased cells but not in the matching normal cells. Conversely, the disease may be treated by reducing the expression of one or more miRNA known to be consistently overexpressed in the diseased cells but not in the matching normal cells by, for example, antagomirs of the overexpressed miRNAs.

Diseases treatable by the subject invention include, for instance, but are not limited to, cancer, infectious disease, cystic fibrosis, blood disorders, including leukemia and lymphoma, spinal muscular dystrophy, early-onset Parkinsonism (Waisman syndrome) and X-linked mental retardation (MRX3).

Cancers include but are not limited to biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

An infectious disease, as used herein, is a disease arising from the presence of a foreign microorganism in the body. A microbial antigen, as used herein, is an antigen of a microorganism. Microorganisms include but are not limited to, infectious virus, infectious bacteria, and infectious fungi.

Examples of infectious virus include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Examples of infectious bacteria include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of infectious fungi include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium such as Plasmodium falciparum, Plasmodium malariae, Plasmodium ovale, and Plasmodium vivax and Toxoplasma gondii.

The vectors of this invention can be delivered into host cells via a variety of methods, including but not limited to, liposome fusion (transposomes), infection by viral vectors, and routine nucleic acid transfection methods such as electroporation, calcium phosphate precipitation and microinjection. In some embodiments, the vectors are integrated into the genome of a transgenic animal (e.g., a mouse, a rabbit, a hamster, or a nonhuman primate). Diseased or disease-prone cells containing these vectors can be used as a model system to study the development, maintenance, or progression of a disease that is affected by the presence or absence of the interfering RNA.

Expression of the miRNA/siRNA introduced into a target cell may be confirmed by art-recognized techniques, such as Northern blotting using a nucleic acid probe. For cell lines that are more difficult to transfect, more extracted RNA can be used for analyses, optionally coupled with exposing the film longer. Once expression of the miRNA/siRNA is confirmed, the DNA construct can then be tested for RNAi efficacy against a cotransfected construct encoding the target protein or directly against an endogenous target. In the latter case, one preferably should have a clear idea of transfection efficiency and of the half-life of the target protein before performing the experiment.

### VIII. Pharmaceutical Use and Methods of Administration

In one aspect, the invention provides a method of administering any of the compositions described herein to a subject. When administered, the compositions are applied in a therapeutically effective, pharmaceutically acceptable amount as a pharmaceutically acceptable formulation. As used herein, the term “pharmaceutically acceptable” is given its ordinary meaning. Pharmaceutically acceptable compounds are generally compatible with other materials of the formulation and are not generally deleterious to the subject. Any of the compositions of the present invention may be administered to the subject in a therapeutically effective dose. A “therapeutically effective” or an “effective” as used herein means that amount necessary to delay the onset of, inhibit the progression of, halt altogether the onset or progression of, diagnose a particular condition being treated, or otherwise achieve a medically desirable result, i.e., that amount which is capable of at least partially preventing, reversing, reducing, decreasing, ameliorating, or otherwise suppressing the particular condition being treated. A therapeutically effective amount can be determined on an individual basis and will be based, at least in part, on consideration of the species of mammal, the mammal's age, sex, size, and health; the compound and/or composition used, the type of delivery system used; the time of administration relative to the severity of the disease; and whether a single, multiple, or controlled-release dose regiment is employed. A therapeutically effective amount can be determined by one of ordinary skill in the art employing such factors and using no more than routine experimentation.

When administered to a subject, effective amounts will depend on the particular condition being treated and the desired outcome. A therapeutically effective dose may be determined by those of ordinary skill in the art, for instance, employing factors such as those further described below and using no more than routine experimentation.

In administering the systems and methods of the invention to a subject, dosing amounts, dosing schedules, routes of administration, and the like may be selected so as to affect known activities of these systems and methods. Dosage may be adjusted appropriately to achieve desired drug levels, local or systemic, depending upon the mode of administration. The doses may be given in one or several administrations per day. As one example, if daily doses are required, daily doses may be from about 0.01 mg/kg/day to about 1000 mg/kg/day, and in some embodiments, from about 0.1 to about 100 mg/kg/day or from about 1 mg/kg/day to about 10 mg/kg/day. Parental administration, in some cases, may be from one to several orders of magnitude lower dose per day, as compared to oral doses. For example, the dosage of an active compound when parentally administered may be between about 0.1 micrograms/kg/day to about 10 mg/kg/day, and in some embodiments, from about 1 microgram/kg/day to about 1 mg/kg/day or from about 0.01 mg/kg/day to about 0.1 mg/kg/day.

In some embodiments, the concentration of the active compound(s), if administered systemically, is at a dose of about 1.0 mg to about 2000 mg for an adult of 70 kg body weight, per day. In other embodiments, the dose is about 10 mg to about 1000 mg/70 kg/day. In yet other embodiments, the dose is about 100 mg to about 500 mg/70 kg/day. Preferably, the concentration, if applied topically, is about 0.1 mg to about 500 mg/gm of ointment or other base, more preferably about 1.0 mg to about 100 mg/gm of base, and most preferably, about 30 mg to about 70 mg/gm of base. The specific concentration partially depends upon the particular composition used, as some are more effective than others. The dosage concentration of the composition actually administered is dependent at least in part upon the particular physiological response being treated, the final concentration of composition that is desired at the site of action, the method of administration, the efficacy of the particular composition, the longevity of the particular composition, and the timing of administration relative to the severity of the disease. Preferably, the dosage form is such that it does not substantially deleteriously affect the mammal. The dosage can be determined by one of ordinary skill in the art employing such factors and using no more than routine experimentation.

Any medically acceptable method may be used to administer a composition to the subject. The administration may be localized (i.e., to a particular region, physiological system, tissue, organ, or cell type) or systemic, depending on the condition being treated. For example, the composition may be administered orally, vaginally, rectally, buccally, pulmonary, topically, nasally, transdermally, through parenteral injection or implantation, via surgical administration, or any other method of administration where suitable access to a target is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be preferred in some embodiments because of the convenience to the subject as well as the dosing schedule. Compositions suitable for oral administration may be presented as discrete units such as hard or soft capsules, pills, cachettes, tablets, troches, or lozenges, each containing a predetermined amount of the active compound. Other oral compositions suitable for use with the invention include solutions or suspensions in aqueous or non-aqueous liquids such as a syrup, an elixir, or an emulsion. In another set of embodiments, the composition may be used to fortify a food or a beverage.

In some embodiments, the compositions of the invention may include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof, or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

### EXAMPLES

The invention now being generally described, it will be more readily understood by reference to the following examples, which are included merely for purposes of illustration of certain aspects and embodiments of the present invention, and are not intended to limit the invention.

### Introduction

According to the instant invention, microRNA expression profiles are often characteristic of specific cell-types. The following examples describe the characterization of microRNA expression profiles in several specific cell lines, such as the mouse mammary epithelial cell line Comma-Dβwhich contains a population of self-renewing progenitor cells that can reconstitute the mammary gland.

Specifically, Applicants have purified this population and determined its microRNA expression profile/signature. Several microRNAs, including miR-205 and miR-22, are highly expressed in mammary progenitor cells, while others, including let-7 and miR-93, are depleted. Let-7 sensors can be used to prospectively enrich self-renewing populations, and enforced let-7 expression induces loss of self-renewing cells from mixed cultures.

Overall, these results support the notion that miRNA expression patterns form both a characteristic signature of a given cell type and help to reinforce cell fate specification. Even within a single cell line, distinct compartments containing progenitor cells and more differentiated cells have unique miRNA patterns, suggesting that such signatures can be used not only to define and track rare cell populations in vitro and in vivo, but that manipulation of these signatures might be used to expand or deplete stem cell and tumor initiating cell populations for therapeutic benefit.

### Example I

### ALDH is a Marker of Mammary Progenitor Cells

In many tissues, stem and progenitor cell populations are becoming increasingly well defined. In the mammary gland, this was elegantly demonstrated by the reconstitution of a functional gland from a single stem cell, which was isolated using cell surface markers, CD49f, CD29, and CD24 (Shackelton et al., 2006, Stingl et al., 2006). Hematopoietic stem cells and neuronal progenitor cells have also been isolated on the basis of ALDH activity (Hess et al., 2006, Corti et al., 2006). Interestingly, ALDH positive cells derived from AML patients have increased NOD/SCID engraftment potential relative to ALDH negative cells, suggesting that these cells represent primitive leukemic stem cells (Cheung et al., 2007).

Comma-Dβ cells harbor a permanent population of undifferentiated basal cells that are able to reconstitute the mammary tree (Deugnier et al., 2006). Applicants realize that these cells provide an excellent system in which to study the role of miRNAs in stem cell maintenance, self-renewal and differentiation. By combining ALDH and Sca-1 (stem cell antigen) expression criteria, Applicants performed an unbiased characterization of miRNAs in mammary progenitor populations using deep sequencing. These studies identified miRNAs that are highly expressed in the progenitor fraction as well as miRNAs that are relatively depleted in this population. By manipulating expression of at least one of these miRNAs, Applicants linked miRNAs to progenitor self-renewal.

Sca-1^{high} Comma-Dβ cells have retained the ability to reconstitute a functional mammary gland upon transplantation of as few as 1000 cells into the fat pad of a syngeneic virgin female (Deugnier et al., 2006). 2-D and 3D cultures, including mammosphere assays, have provided evidence of the self-renewal and differentiation capacity of these cells as they can generate both myoepithelial and luminal cells in vitro (Deugnier et al., 2006, Chen et al., 2007).

Since Sca-1 expression was not enriched in the recently defined murine mammary stem/progenitor cells (Shackleton et al., 2006), we asked whether ALDH expression could be used to isolate progenitor populations from Comma-Dβ. Applicants also tested whether a combination of ALDH and Sca-1 markers provided increased specificity for progenitor cells, at least in cultured populations.

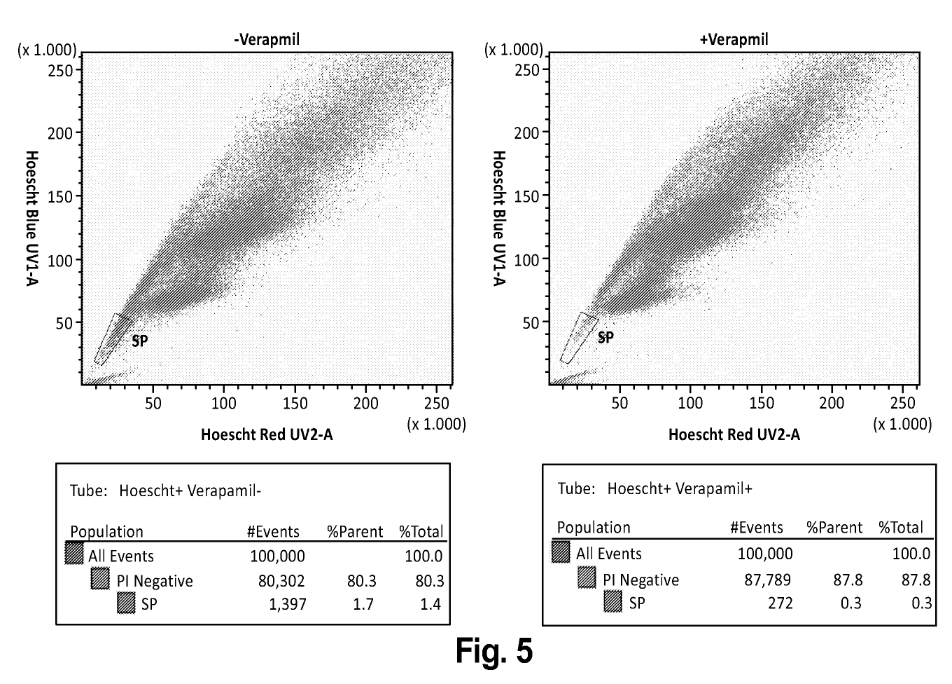

ALDH activity can be measured in living cells by using a fluorogenic substrate, ALDEFLUOR (Corti et al., 2006, Hess et al., 2006). ALDH induces retention of this substrate, resulting in increased florescence. Truly positive cells can be identified by comparison to cells cultured in ALDEFLUOR in the presence of DEAB, an ALDH inhibitor. The Comma-Dβ cell line contains ALDH^{bright} Sca-1^{high} cells that comprise 2% of the total population (FIG. 1A). This number is consistent with the number of side population (SP) cells we observed in this cell line (FIG. 5).

Colony formation on irradiated feeders or Matrigel is commonly used to assess the proliferative capacity of purified epithelial stem and progenitor cells. In several studies, this capacity has been shown to correlate with in vivo morphogenic potential (Shackelton et al., 2006, Deugnier et al., 2006). Applicants therefore examined the colony forming capacity of four sorted populations (ALDH^{bright} Sca-1^{high}, ALDH^{bright} Sca-1^{neg}, ALDH^{neg} Sca-1^{high}, & ALDH^{neg} Sca-1^{neg}).

Only the two ALDH^{bright} populations yielded significant numbers of colonies, with the ALDH^{bright} Sca-1^{high} subset exhibiting a 3-fold greater colony-forming frequency and substantially larger colonies (FIG. 1B). ALDH^{bright} cells gave rise to both luminal and myoepithelial colonies, based on morphology (FIG. 1C). A third colony morphology was also observed that fit neither the dispersed tear-drop shape characteristic of myoepithelial cells nor the tightly arranged cells with distinct cell borders that indicate luminal cells (Stingl et al., 1998).

ALDH bight Sca-1^{high} cells plated at clonogenic density in Matrigel expanded and formed spheroids (avg. 46/well n=4) (p<0.001), whereas the ALDH^{bright} Sca-1^{neg} cells grew poorly under these conditions (FIG. 1D). ALDH^{neg} cells were unable to form colonies. These results are consistent with previous studies showing the inability of Sca-1^{neg} cells to grow in Matrigel (Deugnier et al., 2006).

### Example II Alternative Methods for Isolation/Enrichment of ALDH-Positive Cells

Resistance to a group of anticancer drugs called oxazaphosphorines has been linked to ALDH activity (Bunting et al., 1996). Applicants reasoned that mafosfamide (MAF) treatment might enrich the population of ALDH^{bright} progenitor cells. Thus Applicants treated cells for four days and analyzed the surviving population by FACS. This resulted in a 15-fold enrichment in ALDH bight Sca-1^{high} cells. Thus, the progenitor population resident within Comma-Dβ can be selected by this method, and these progenitors are intrinsically resistant to at least some anti-cancer drugs (FIGS. 1E and 1F).

Following selection, Applicants also noted a 2-fold expansion in the ALDH^{neg} Sca-1^{high} compartment. It is possible that the apparent expansion arise from differentiation of selected ALDH^{bright} Sca-1^{high} cells, or alternativey, the resistance of this population to MAF.

MAF is a cyclophosphamide derivative that is active in cultured cells. Cyclophosphamide is commonly used as part of a first-line therapy for breast cancer (Smith et al., 2003). Thus, the finding that treatment with MAF can enrich ALDH-positive cells has profound implications.

### Example III

### An miRNA Fingerprint of Mouse Mammary Epithelial Progenitors

To probe potential roles for miRNAs in the maintenance and differentiation of mammary epithelial progenitor cells, Applicants constructed small RNA libraries from Sca-1^{high}, Sca-1^{neg}, ALDH^{bright} Sca-1^{high}, and MAF-treated Comma-Dβ cells. These were deeply sequenced on the Illumina 1G platform and mapped to the mouse genome using a customized bioinformatics pipeline. Reads were annotated by BLAT (Kent, 2002, incorporated by reference) to a unified database comprised of mouse entries from miRbase (Griffiths-Jones et al., 2006), NONCODE (Liu et al, 2005), tRNAs in “The RNA Modification Database” (Limbach et al., 1994), and rRNA entries in the Entrez Nucleotide Database.

Approximately 50% of all sequences that mapped to the genome corresponded to known miRNAs (Table 1) for Sca-1^{high}, ALDH^{bright} Sca-1^{high} and Sca-1^{neg} libraries

TABLE 1
Distribution of sequencing results for each compartment

| | Condition | | |
|---|---|---|---|
| | | Sca High/ALDH | |
| Name | Sca-1 Negative | bright | MAF |
| Total number of successful | 4,099,736 | 2,270,791 | 1,860,259 |
| Solexa reads: | | | |

Biological Products:

| | | | | | | |
|---|---|---|---|---|---|---|
| miRNA | 2,2059,799 | (54%) | 1,067,613 | (47%) | 1,472,429 | (79%) |
| mRNAlike | 474,384 | (12%) | 255,991 | (11%) | 221,731 | (12%) |
| tRNA | 36,719 | (0.9%) | 11,849 | (0.5%) | 4,949 | (0.3%) |
| piRNA | 1,8825 | (0.46%) | 5,653 | (0.25%) | 4,886 | (0.26%) |
| rRNA | 1,250 | (<0.1%) | 1,109 | (<0.1%) | 282 | (<0.1%) |
| snoRNA | 504 | (<0.1%) | 192 | (<0.1%) | 140 | (<0.1%) |
| snRNA | 47 | (<0.1) | 58 | (<0.1%) | 0 | (0%) |
| Other RNAs | 2,646 | (<0.1%) | 420 | (<0.1%) | 454 | (<0.1%) |

Technical Artifacts:

| | | | | | | |
|---|---|---|---|---|---|---|
| Adaptor self-ligation | 134,510 | (3.28%) | 159,210 | (7.01%) | 17,444 | (0.93%) |
| Spiked-in radio-labeled | 60,873 | (1.48%) | 295,279 | (1.30%) | 47,906 | (2.57%) |
| RNA marker | | | | | | |

Undefined:

| | | | | | | |
|---|---|---|---|---|---|---|
| Undefined | 1,163,999 | (28.39%) | 473,417 | (20.84%) | 90,038 | (4.8%) |

Note:
All the successful Solexa reads were compared using BLAT (Kent, W. J. BLAT - The BLAST-Like Alignment Tool. Genome Res. 12(4), 656-664 (2002)) to a database that was comprised of mouse mature miRNA from miRBase (miRBase: microRNA sequences, targets and gene nomenclature. Griffiths-Jones S, Grocock R J, van Dongen S, Bateman A, Enright A J. NAR, 2006, 34, Database Issue, D140-D144), mouse non-coding RNA from NONCODE (NONCODE: an integrated knowledge database of non-coding RNAs Nucleic Acids Research, 2005, Vol. 33, Database issue D112-D115), mouse tRNA from (Limbach P. A., Crain P. F., McCloskey. J. A. 1994. Summary: the modified nucleosides of RNA.

Nucleic Acids Res. 22: 2183-2196). Undefined represents the class of sequences that could not be annotated using this database.

In the MAF library, 80% of reads mapped to miRNAs. Breakdown products of noncoding RNAs such as rRNAs, tRNAs, snRNAs, snoRNAs, and others represented less than 0.5% of total sequences for all four libraries. An estimated 25% of sequences mapped neither to known miRNAs nor other annotated small RNAs in the sorted libraries whereas only 5% remained unidentified for the MAF library. The top 50 miRNAs sorted based on abundance in the ALDH^{bright} Sca-1^{high} library are shown in Table 2.

TABLE 2
The 50 most abundant differentially expressed
microRNAs cloned from the four distinct libraries sorted by
abundance in ALDH^{br}Sca^{hi} library

| Name | Sca⁻ | Sca⁺ | Sca⁺/ALDH⁺ | MAF |
|---|---|---|---|---|
| mmu-miR-205 | 19863 | 245719 | 282099 | 68275 |
| mmu-miR-21 | 481446 | 983326 | 194865 | 472852 |
| mmu-miR-22 | 53768 | 177050 | 140987 | 131022 |
| mmu-miR-31 | 70341 | 350889 | 86879 | 138207 |
| mmu-let-7c | 252067 | 151885 | 67186 | 134301 |
| mmu-miR-29a | 35707 | 79858 | 59601 | 36501 |
| mmu-let-7b | 350721 | 136707 | 44986 | 73731 |
| mmu-miR-24 | 76273 | 194739 | 39141 | 76414 |
| mmu-miR-29b | 29736 | 69223 | 31442 | 45792 |
| mmu-let-7a | 42194 | 68022 | 25015 | 41828 |
| mmu-let-7f | 23513 | 58940 | 22726 | 40008 |
| mmu-miR-130a | 31343 | 32538 | 20878 | 36732 |
| mmu-miR-143 | 169575 | 107243 | 18784 | 13747 |
| mmu-let-7i | 30523 | 27783 | 18424 | 25134 |
| mmu-miR-20a | 112710 | 98273 | 15711 | 31599 |
| mmu-miR-103 | 36344 | 66014 | 14593 | 31678 |
| mmu-miR-93 | 146002 | 90496 | 12521 | 26717 |
| mmu-miR-16 | 6130 | 46814 | 10002 | 21188 |
| mmu-let-7g | 17060 | 27643 | 9857 | 15469 |
| mmu-let-7d | 23400 | 42094 | 8432 | 13661 |
| mmu-miR-30a-5p | 9121 | 13441 | 8238 | 17062 |
| mmu-miR-26a | 11669 | 17514 | 7253 | 14106 |
| mmu-miR-10a | 8238 | 5136 | 7205 | 8316 |
| mmu-let-7e | 9472 | 13042 | 6887 | 7454 |
| mmu-miR-125b | 21591 | 58415 | 6789 | 4243 |
| mmu-miR-221 | 14678 | 37425 | 6499 | 3543 |
| mmu-miR-320 | 11634 | 7985 | 6293 | 5242 |
| mmu-miR-140* | 19716 | 31507 | 5848 | 3780 |
| mmu-miR-92 | 1219 | 7993 | 4265 | 2043 |
| mmu-miR-99b | 5934 | 7434 | 4180 | 4099 |
| mmu-miR-30d | 2963 | 5209 | 3966 | 4423 |
| mmu-miR-210 | 8556 | 4564 | 3932 | 2592 |
| mmu-miR-27b | 21564 | 52185 | 3929 | 4537 |
| mmu-miR-181a | 5993 | 4373 | 3489 | 3961 |
| mmu-miR-99a | 1605 | 2413 | 3454 | 2588 |
| mmu-miR-100 | 2547 | 3668 | 3055 | 3448 |
| mmu-miR-27a | 29974 | 53643 | 3051 | 4661 |
| mmu-miR-652 | 19402 | 10406 | 2997 | 3359 |
| mmu-miR-191 | 5599 | 6619 | 2978 | 6834 |
| mmu-miR-23a | 33020 | 171674 | 2936 | 6008 |
| mmu-miR-200a | 1987 | 1477 | 2691 | 11914 |
| mmu-miR-674 | 7689 | 6755 | 2372 | 3005 |
| mmu-miR-183 | 2800 | 7719 | 2296 | 3091 |
| mmu-miR-218 | 2183 | 4141 | 2187 | 1877 |
| mmu-miR-101b | 3773 | 7742 | 1966 | 1841 |
| mmu-miR-429 | 1788 | 1758 | 1893 | 10349 |
| mmu-miR-23b | 11674 | 104212 | 1739 | 3286 |
| mmu-miR-125a | 3172 | 10349 | 1698 | 827 |
| mmu-miR-26b | 5200 | 6697 | 1635 | 4009 |
| mmu-miR-107 | 3264 | 6585 | 1635 | 2427 |
| Total No. of known | 2,469,404 | 3,980,114 | 1,274,811 | 1,676,774 |
| miRNA* | | | | |
| Total No. of | 4,099,736 | 6,648,439 | 2,270,791 | 1,860,259 |
| successful reads: | | | | |
| Total No. of reads | 4,783,145 | 6,844,356 | 2,433,920 | 2,336,839 |
| with known errors: | | | | |

Data represents raw counts for each miRNA.
*Using BLAT by Kent W J. Parametes: -minIdentity = 90 -minScore = 17 -tileSize = 6 -minMatch = 1
Database: All Mus musculus entries in mature.fa from mirBase v9.2

Expression signatures are often presented as heat maps, illustrating the relative signal for an individual species in two samples. Although there are undoubtedly biases in the cloning of specific RNAs, the available sequence data permitted Applicants to examine both differential expression and approximate abundance. Applicants reasoned that focusing on highly expressed miRNAs would maximize the possibility of identifying those that are biologically relevant. A “bubble plot” can be used to depict both the abundance of a particular miRNA (given as the sum of the reads in the two libraries) and its relative expression (plotted as a log 2 of the ratio of reads in each library).

The ALDH^{bright} Sca-1^{high} (FIG. 2A.) and the MAF libraries (FIG. 2B) were compared to the Sca-1^{neg} library to identify differentially expressed miRNAs. Two abundant miRNAs, miR-205 and miR-22, were consistently enriched in the progenitor population. Both were also abundant in Sca-1^{high} library, suggesting that they may be important for the basic physiology or identity of basal cells.

MiRNA expression profiling of various tissues showed that miR-205 was preferentially expressed in breast and thymus (Baskerville et al., 2005). In human embryonic stem cells, Nanog and Sox2 binding sites are located near the miR-205 and miR-22 promoters (Boyer et al, 2005). However, in comparing our dataset to ES cell-specific miRNAs no consistent overlap in patterns was found.

Other miRNAs showed substantially lower expression in the progenitor compartment. Let-7b, let-7c and miR-93 were the most abundant miRNAs that showed preferential expression in Sca-1neg cells. Collectively, let-7b and let-7c represented only 8.8% of the total miRNA sequences in the ALDH^{bright} Sca-1^{high} library compared to 24% of miRNA sequences in Sca-1^{neg} cells. Interestingly, miR-20a is part of a polycistronic cluster containing 17-5p, miR-18a, miR-19b.

These are also underrepresented in the progenitor compartment. miR-21 was the most abundant miRNA found in relatively equal amounts in all four libraries constituting a consistent average of 30% of mapped miRNAs sequences. Overall, the trends in miRNA representation seen upon comparison of ALDH^{bright} Sca-1^{high} to Sca-1^{neg} cells were reproduced upon examination of the MAF-treated library. However, miR-200a and miR-429, both of which are part of miR-8 family, were found at substantial levels in the MAF library only.

Applicants performed an independent verification of differential miRNA expression using quantitative stem-loop PCR (qRT-PCR) as previously described (Chen et al., 2005) (FIG. 2C). Applicants examined expression of let-7b, let-7c, miR-93, miR-23b, miR-23a, miR-205, miR-31 in the Sca-1^{high} fraction vs Sca-1^{neg} libraries.

In all 7 cases, differential expression was confirmed, though the absolute magnitudes of expression ratios did not precisely agree with those determined from sequencing data.

### Example IV

### let-7/miR-93 Depletes the Self-renewing ALDH Compartment in Comma-Dβ

Applicants investigated a role for reduced let-7 expression in mammary progenitor cells. First, it was necessary to investigate whether the ALDH^{bright} Sca-1^{high} compartment was receptive to signals known to expand stem/progenitor populations and whether miRNA expression patterns responded similarly. Enforced expression of β-catenin in Comma-Dβ cells was shown to expand the Sca-1^{high} compartment and increase mammosphere-forming capacity (Chen et al, 2007). Similarly, Applicants observed a 3.5-fold increase in the ALDH^{bright} Sca-1^{high} population upon the ectopic expression of Wnt-1 (FIG. 3A).

Wnt-1-expressing cells survived higher doses of MAF than empty vector control cells (FIG. 3B), consistent with the ALDH^{bright} Sca-1^{high} compartment having intrinsically higher drug resistance.

In concert with changes in the progenitor compartment, we observed that Wnt-1-expressing cells expressed 6-fold higher levels of miR-205 when compared to empty vector control cells with no observed reduction in let-7b, let-7c, or miR-93 expression, as might be expected since the differentiated compartments were still prominent in this mixed population (data not shown).

To probe the functional relevance of differential miRNA expression patterns, Applicants examined the consequences of enforced expression of let-7c. Comma-Dβ let-7c cells showed a substantial, 6-fold reduction in the ALDH^{bright} compartment (n=4). In concert, Applicants observed the emergence of distinctly Sca-1^{neg} and Sca-1^{lo} populations (FIG. 3C).

Similar results are also obtained by enforced expression of miR-93 (FIG. 3D).

These results suggest that differences in miRNA expression between differentiated and self-renewing populations within Comma-Dβ cells have substantial impacts on cell identity and physiology.

### Example V

### A let-7 Sensor Marks the Progenitor Compartment

Convenient markers for rare cell populations have proven difficult to identify. miRNA sensors have been used in plants and animals to visualize the expression patterns of individual small RNA species. Applicants demonstrated the general principle here that miRNA sensors, as directed by our observed expression patterns, could be used to mark rare cell populations and permit their isolation.

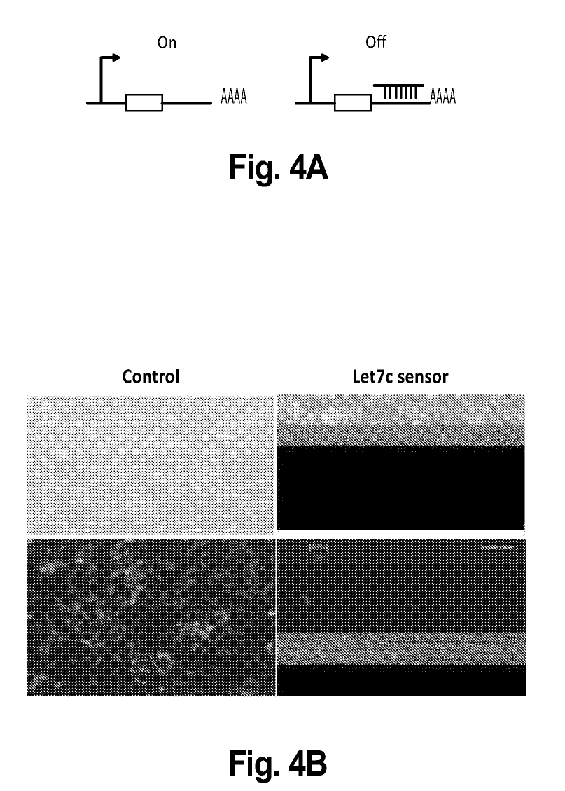

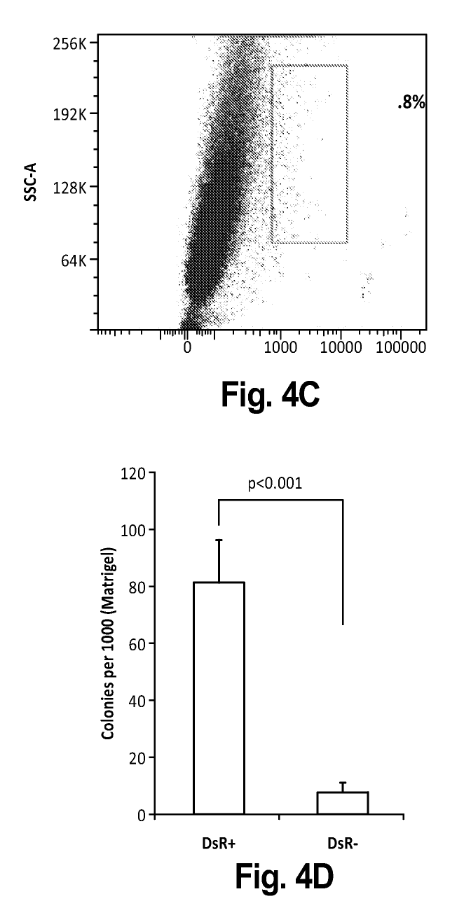

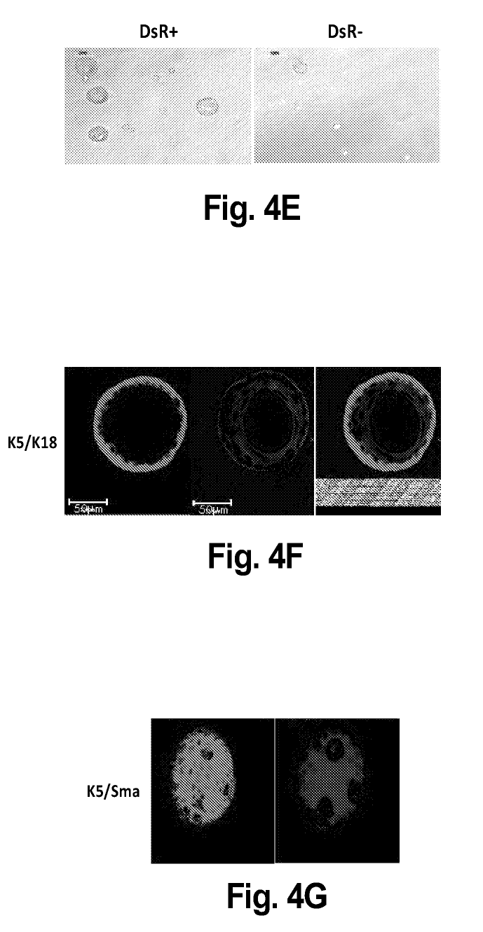

Applicants first constructed a let-7c sensor by introducing its perfect complement into the 3′ untranslated region of DsRed, thus specifying silencing by RNAi in the presence of the miRNA (FIG. 4A).

Since let-7c expression is low in ALDH^{bright} Sca-1^{high} cells, Applicants predicted that the sensor would not be silenced, thus marking the progenitor compartment by DsRed expression. Where let-7c expression is high in the more differentiated cell types, Applicants predicted that the sensor would be silenced (FIG. 4B). Indeed, Applicants found that overall, DsRed-positive cells (DsR⁺) constituted 0.8% of the population (FIG. 4C). DsRed-positive cells are enriched for Sca-1^{high} and ALDH expressing cells, as expected (data not shown).

Applicants tested DsRed⁺ cells for their ability to self-renew and differentiate in vitro. DsR⁺ cells formed spheroids with 10-fold greater efficacy than DsR⁻ cells (FIG. 4D), with only DsR⁺ cells forming spheroids greater than 50 μm in size (FIG. 4E). Confocal images of spheroids co-stained with Keratin 5 (K5) and Keratin 18 (K18) revealed that a single DsR⁺ cell was able to give rise to a K5-positive, basal, outer layer and an inner layer of luminal, K18-positive cells (FIG. 4F), though, consistent with previous observations, not all spheres had such an apparent luminal structure (Deugnier et al., 2006).

To probe the ability of DsR⁺ cells to differentiate into myoepithelial cells we co-stained spheroids with K5 and smooth muscle actin (SMA) and indeed observed spheroids with an outer layer of K5- and SMA-positive cells (FIG. 4G).

These studies demonstrate that a lack of let-7c expression can be used to prospectively isolate mammary progenitor cells. Perhaps in combination with additional sensors, this allows the experimentally determined miRNA expression signature to be converted into a functional tool that can augment existing markers of murine progenitors and likely also tumor initiating cells.

Overall, our results support the notion that miRNA expression patterns form both a characteristic signature of a given cell type and help to reinforce cell fate specification. Even within a single cell line, distinct compartments containing progenitor cells and more differentiated cells have unique miRNA patterns, suggesting that such signatures can be used not only to define and track rare cell populations in vitro and in vivo but that manipulation of these signatures might be used to expand or deplete stem cell and tumor initiating cell populations for therapeutic benefit.

### Methods

The following methods and reagents were used in the Examples above, or are generally known in the art. These are merely for illustrative purpose, and are by no means limiting. Other comparable minor variations can be readily made without undue experimentation for adapting to specific problems.

### Cell Culture

Comma-Dβ cells were grown in DMEM:F12 (HyClone) supplemented with 2% FCS, 5 ng/ml murine EGF (Sigma), 10 μg/ml human insulin (Sigma), and 50 μg/ml gentamicin (Gibco). Cells were only used within passages 17-35. Phoenix cells were maintained in DMEM supplemented with 10% FBS (Hyclone), and penicillin-streptomycin (Gibco).

### Constructs and Infections

For construction of Let7c stable expression vectors, the following primers were used: forward-5′ GGC CAG ATC TGT GTG GTC AAG GAG ATG TTA G-3′ (SEQ ID NO: 1) and reverse 5′ GAT CCT CGA GTA ACA GCC CGT GAG AAA TAG-3′ (SEQ ID NO: 2) containing Bgl-II/XhoI restriction sites. A 500 bp fragment was PCR amplified from mouse genomic DNA and cloned into an MSCV vector carrying a hygromycin cassette (Clontech). Phoenix cell transfections were performed using LT-1 transfection reagent (Mirus) according to manufacture's instructions. To construct Wnt-1MSCV, the 1.9 kb fragment of wingless cDNA (nucleotides 284-2181) in pMV7 (kind gift of Anthony Brown) was subcloned into an MSCV-hygro vector. For construction of the Let7c sensor, miRNA-complementary oligonucleotides were annealed and cloned into a Marx vector that directs dsRED expression.

### ALDEFLUOR and SP Cell Staining and Flow Cytometry

Cells were stained at 10⁶ cells/ml in assay buffer containing 1 μmole BAAA for 1 hr at 37° C. The ALDEFLUOR kit was purchased from StemCell Technologies (Durham, N.C., USA). An aliquot of stained cells were treated with 50 mmol/L DEAB as a negative control. After ALDEFLUOR staining, cells were co-stained with anti-Sca-1-PE (BD Pharmigen) for 20 minutes on ice. For small RNA cloning cells were FACS sorted directly into Trizol LS reagent (Invitrogen). ALDEFLUOR was excited at 488 nm, and fluorescence emission was detected using a standard fluorescein isothiocyanate (FITC) 530/30-nm band-pass filter. For SP analysis, cells were stained with Hoescht 33342 Dye as previously described (Goodell et al., 1996).

### In Vitro Assays

Colony formation assays on feeders were essentially performed as described by Shackleton et al 2006 (incorporated by reference). Three dimensional (3D) cultures were performed as described in (Debnath et al., 2003, incorporated by reference).

### Antibodies and Immunofluorescence

The following primary antibodies were used: anti-Sca-1 PE (BD pharmigen); mouse anti-cytokertain peptide 18 (Sigma), mouse anti-a-Sma (Sigma), rabbit polyclonal anti-cytokeratin 5 (Covance). Fluorochrome-conjugated secondary antibodies included anti-rabbit IgG-Alexa488 and anti-mouse IgG-Alexa647 (Molecular Probes).

### Small RNA Cloning

1-4 μg of total RNA from sorted cells was used for small RNA cloning performed as described in Pfeffer et al, 2005 (incorporated by reference). Illumina 1G sequencing and analysis was performed as described Stark et al. 2007 (incorporated by reference). miRNA expression analyses mature miRNAs were quantified using the TaqMan MicroRNA Assays previously described by Chen et al., 2005 (Applied Biosystems, incorporated by reference).

Data was normalized to Actin using SuperScript III SYBR Green One-Step qRT-PCR system (Invitrogen). The experiments were repeated twice and all reactions were run in triplicate.

### Cell Viability Assay

To assess the cytotoxic effects of MAF cells were seeded at 5,000 cells per well in a 96-well format. 24 h or 48 h later cells were treated with various doses of mafosfamide L-lysine salt (D-17930) (NIOMECH in der IIT GmbH) freshly dissolved in water. Cell viability was measured using CellTiter-Glo® Luminescent Cell Viability Assay (PROMEGA).

Citations (including manufacture recommendation and protocols) relied upon for the experiments described herein are incorporated herein by reference.

### Mammosphere Assay

Non-adherent mammospheres are an in vitro culture system that allows for the propagation of primary human mammary epithelial stem and progenitor cells in an undifferentiated state, based on their ability to proliferate in suspension as spherical structures. Non-adherent mammospheres have previously been described in Dontu et al. (Genes Dev. 2003 May 15; 17(10): 1253-70), and Dontu et al. (Breast Cancer Res. 2004; 6(6):R605-15). These references are incorporated by reference in their entireties and specifically for teaching the construction and use of non-adherent mammospheres. As described in Dontu et al., mammospheres have been characterized as being composed of stem and progenitor cells capable of self-renewal and multi-lineage differentiation. Dontu et al. also describes that mammospheres contain cells capable of clonally generating complex functional ductal-alveolar structures in reconstituted 3-D culture systems in Matrigel.

In an exemplary nonadherent mammosphere assay, large ducts, terminal ducts (identified by connecting alveoli), and lobules are isolated and trypsinized for 10-15 min at 37° C. on an orbital shaker to obtain a single cell suspension. Nonadherent mammosphere cultures were prepared as previously described (Dontu et al., In vitro propagation and transcriptional profiling of human mammary stem/progenitor cells. Genes Dev. 17: 1253-1270, 2003, incorporated herein by reference). In brief, cells are plated at a concentration of 5,000-20,000 cells/ml. The cultures are monitored for up to 12 days for the appearance of mammospheres. After 8 days, cultures are photographed, and structures derived from ducts (large and terminal) and lobules, respectively, are quantified and separated into two categories: >70 μm and <70 μm (n=3×200 structures). For analysis of keratin expression, duct- and lobule-derived mammospheres are either smeared onto a glass slide and stained or trypsinized at day 9, plated at clonal density (200 cells/cm²), and propagated for 5 days in F12 medium before immunocytochemical staining. Colonies from each segment are quantified using a fluorescence microscope (Dialux 20; Leitz) equipped with a 10× objective. Mammosphere populations derived from ducts and lobules are assessed for morphogenic potential by inoculation for 3 wk of each population in 300 μl lrECM (Matrigel; Becton Dickinson). Some cultures are conditioned by a feeder layer of primary human breast epithelial cells separated from the top gel by 200 μl of cell-free gel. The number of mammosphere-derived budding structures is assessed by phase-contrast microscopy.

The practice of aspects of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

All patents, patent applications and references cited herein are incorporated in their entirety by reference.

### LITERATURE CITED

Baskerville, S., Bartel, D. P. Microarray profiling of microRNAs reveals frequent coexpression with neighboring miRNAs and host genes. 2005. RNA 11:241-247.
Bernstein, E., Kim , S. Y., Carmell, M. A., Murchison, E. P., Alcorn, H., Li, M. Z., Mills, A. A., Elledge, S. J., Anderson K. V., Hannon G. J. 2003. Dicer is essential for mouse development. Nat. Genet. 35: 215-217.
Boyer, L. A., Lee, T. I., Cole, M. F., Johnstone, S. E., Levine, S. S., Zucker, J. P., Guenther, M. G., Kumar, R. M., Murray, H. L., Jenner, R. G., Gifford, D. K., Melton, D. A., Jaenisch, R., Young, R. A. 2005. Core transcriptional regulatory circuitry in human embryonic stem cells. Cell 122:947-56.
Bunting, K. D., Townsend, A. J. 1996. De novo expression of transfected human class 1 aldehyde dehydrogenase (ALDH) causes resistance to oxazaphosphorine anti-cancer alkylating agents in hamster V79 cell lines. Elevated class 1 ALDH activity is closely correlated with reduction in DNA interstrand cross-linking and lethality. J Biol. Chem. 27:11884-11890.
Chen C, Ridzon, D. A., Broomer, A. J., Zhou, Z, Lee, D. H., Nguyen, J. T., Barbisin, M, Xu, N. L., Mahuvakar, V. R., Andersen, M. R., Lao, K. Q., Livak, K. J., Guegler, K. J. 2005. Real-time quantification of microRNAs by stem-loop RT-PCR. Nucleic Acids Res. 33: e179.
Chen, M. S., Woodward, W. A., Behbod, F., Peddibhotla, S., Alfaro, M. P., Buchholz, T. A., Rosen, J. M. 2007. Wnt/beta-catenin mediates radiation resistance of ScaI+progenitors in an immortalized mammary gland cell line. J Cell Sci. 120:468-77.
Cheung, A. M., Wan, T. S., Leung, J. C., Chan, L. Y., Huang, H., Kwong, Y. L., Liang, R., Leung, A. Y. 2007. Aldehyde dehydrogenase activity in leukemic blasts defines a subgroup of acute myeloid leukemia with adverse prognosis and superior NOD/SCID engrafting potential. Leukemia 21:1423-30.
Corti, S., Locatelli, F., Papadimitriou, D., Donadoni, C., Salani, S., Del Bo, R., Strazzer, S., Bresolin, N., Comi, G. P. Identification of a primitive brain-derived neural stem cell population based on aldehyde dehydrogenase activity. 2006. Stem Cells 24:975-85.
Debnath, J., Muthuswamy, S. K. & Brugge, J. S. 2003. Morphogenesis and oncogenesis of MCF-10A mammary epithelial acini grown in three-dimensional basement membrane cultures. Methods 30: 256-268.
Deugnier, M. A., Faraldo, M. M., Teuliere, J., Thiery, J. P., Medina, D., Glukhova, M. A. 2006. Isolation of mouse mammary epithelial progenitor cells with basal characteristics from the Comma-Dbeta cell line. Dev Biol. 293:414-25.
Förstemann, K., Tomari, Y., Du, T., Vagin, V. V., Denli, A. M., Bratu, D. P., Klattenhoff, C., Theurkauf, W. E., Zamore, P. D. Normal microRNA maturation and germ-line stem cell maintenance requires Loquacious, a double-stranded RNAbinding domain protein. 2005. PLoS Biol. 3:236.
Giraldez, A. J., Mishima, Y., Rihel, J., Grocock, R. J., Van, Dongen, S., Inoue, K., Enright, A. J., Schier, A. F. 2006. Zebrafish MiR-430 promotes deadenylation and clearance of maternal mRNAs. Science 312:75-9.
Goodell, M. A., Brose, K., Paradis, G., Conner, A. S., Mulligan, R. C. 1996. Isolation and functional properties of murine hematopoietic stem cells that are replicating in vivo. J Exp Med. 183:1797-806.
Griffiths-Jones, S., Grocock R. J., van Dongen S., Bateman A., Enright A. J. 2006. miRBase: microRNA sequences, targets and gene nomenclature. NAR, 34, Database Issue, D140-D144.
Hess, D. A., Wirthlin, L., Craft, T. P., Herrbrich, P. E., Hohm, S. A., Lahey, R., Eades, W. C., Creer, M. H., Nolta, J. A. Selection based on CD133 and high aldehyde dehydrogenase activity isolates long-term reconstituting human hematopoietic stem cells. 2006. Blood 107:2162-2169.
Houbaviy, H. B., Murray, M. F. and Sharp, P. A. 2003. Embryonic stem cell-specific microRNAs. Dev. Cell 5: 351-358.
Jiang, F., Ye, X., Liu, X., Fincher, L., McKearin, D., Liu, Q. Dicer-1 and R3D1-L catalyze microRNA maturation in Drosophila. 2005. Genes Dev. 19:1674-1679 Jin, Z., Xie, T. Dcr-1 maintains Drosophila ovarian stem cells. 2007. Curr Biol. 17:539-44.
Kent, W. J. BLAT—The BLAST-Like Alignment Tool. 2002. Genome Research 4: 656-664.
Lagos-Quintana, M., Rauhut, R., Yalcin, A., Meyer, J., Lendeckel, W., and Tuschl, T. 2002. Identification of tissue-specific microRNAs from mouse. Curr. Biol. 12: 735-739.
Limbach P. A., Crain P. F., McCloskey. J. A. 1994. Summary: the modified nucleosides of RNA. Nucleic Acids Res. 22: 2183-2196.
Liu C., Bai B., Skogerbo G., Cai L., Deng W., Zhang Y., Bu D., Zhao Y., Chen R. 2005. NONCODE: an integrated knowledge database of non-coding RNAs. Nucleic Acids Res. 33 (Database issue):D112-5.
Neilson, J. R., Zheng, G. X., Burge, C. B., Sharp, P. A. Dynamic regulation of miRNA expression in ordered stages of cellular development. 2007. Genes Dev. 21:578-89.
Pfeffer, S., Sewer, A., Lagos-Quintana, M., Sheridan, R., Sander, C., Grasser, F. A., van Dyk, L. F., Ho, C. K., Shuman, S., Chien, M. 2005. Identification of microRNAs of the herpesvirus family. Nat. Methods 2: 269-276.
Reinhart, B. J., Slack, F. J., Basson, M., Pasquinelli, A. E., Bettinger, J. C., Rougvie, A. E., Horvitz, H. R., Ruvkun, G. The 21-nucleotide let-7 RNA regulates developmental timing in Caenorhabditis elegans. 2000. Nature 403:901-6.
Shackleton, M., Vaillant, F., Simpson, K. J., Stingl, J., Smyth, G. K., Asselin-Labat, M. L., Wu, L., Lindeman, G. J., Visvader, J. E. 2006. Generation of a functional mammary gland from a single stem cell. Nature 439:84-8.
Smith, R. E., Bryant, J., DeCillis, A., Anderson, S; National Surgical Adjuvant Breast and Bowel Project Experience. 2003. Acute Myeloid Leukemia and Myelodysplastic Syndrome After Doxorubicin-Cyclophosphamide Adjuvant Therapy for Operable Breast Cancer The National Surgical Adjuvant Breast and Bowel Project Experience Journal of Clinical Oncology 21:1195-1204.
Stark, A., Kheradpour, P., Parts, L., Brennecke, J., Hodges, E., Hannon, G. J., Kellis, M. 2007. Systematic Discovery and characterization of fly microRNA using Drosophilia genomes. Genome Research in Press.
Stingl, J., Eirew, P., Ricketson, I., Shackleton, M., Vaillant, F., Choi, D., Li, H. I., Eaves, C. J.
Purification and unique properties of mammary epithelial stem cells. 2006. Nature 439:993-997.
Stingl, J., Eaves, C. J., Kuusk, U., Emerman, J. T. 1998. Phenotypic and functional characterization in vitro of a multipotent epithelial cell present in the normal adult human breast. Differentiation 63:201-213.
Wang, Y., Medvid, R., Melton, C., Jaenisch, R., Blelloch, R. 2007. DGCR8 is essential for microRNA biogenesis and silencing of embryonic stem cell selfrenewal. Nat. Genet. 39:380-385.

### EQLUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of isolating mammary progenitor cells from a population of mammary cells in culture, the method comprising:
a) introducing into the population of mammary cells an expression cassette comprising (i) a first nucleotide sequence encoding a reporter, and (ii) a second nucleotide sequence complementary to about 12-25 contiguous nucleotides of let-7b, let-7c, or miR-93, wherein the presence of let-7b, let-7c, or miR-93 in a cell inhibits expression of the reporter in the cell; and,
b) isolating cells that do not express the reporter;
thereby isolating mammary progenitor cells.

2. The method of claim 1, wherein the population of mammary cells is from a mammary epithelial cell line or a non-adherent mammosphere.

3. The method of claim 1, wherein the expression cassette is introduced by transfection.

4. The method of claim 1, wherein the expression cassette is introduced by infection.

5. The method of claim 4, wherein the expression cassette further comprises a 5′ LTR, a 3′ LTR, and a viral packaging signal.

6. The method of claim 1, wherein the reporter is a fluorescent protein.

7. The method of claim 1, wherein the reporter is a toxin.

8. The method of claim 1, wherein the second nucleotide sequence is at least 19 nucleotides in length.

9. The method of claim 1, wherein the second nucleotide sequence is located in an untranslated region of the first nucleotide sequence.

10. The method of claim 1, wherein the second nucleotide sequence is perfectly complementary to let-7b, let-7c, or miR-93.

11. The method of claim 1, wherein the expression cassette comprises a nucleotide sequence complementary to about 12 to 23 contiguous nucleotides of at least two miRNAs selected from the group consisting of let-7b, let-7c, and miR-93.

12. A method of isolating mammary progenitor cells from a population of mammary cells in culture, the method comprising:
a) introducing into the population of mammary cells an expression cassette comprising (i) a first nucleotide sequence encoding a reporter, and (ii) a second nucleotide sequence complementary to about 12-25 contiguous nucleotides of miR-205 or miR-22 in a cell inhibits expression of the reporter in the cell, wherein the presence of miR-205 or miR-22 in a cell inhibits expression of the reporter in the cell; and,
b) isolating cells that express the reporter;
thereby isolating mammary progenitor cells.

13. A method of identifying mammary progenitor cells in a population of mammary cells, the method comprising:
a) introducing into the population of mammary cells an expression cassette comprising (i) a first nucleotide sequence encoding a reporter, and (ii) a second nucleotide sequence complementary to about 12-25 contiguous nucleotides of let-7b, let-7c, or miR-93, wherein the presence of let-7b, let-7c, or miR-93 in a cell inhibits expression of the reporter in the cell; and,
b) identifying cells that do not express the reporter;
thereby identifying mammary progenitor cells.

14. The method of claim 13, wherein the expression cassette comprises a tissue-specific promoter, a developmental stage specific promoter, or an inducible promoter.

15. The method of claim 13, wherein cells not expressing the reporter are identified using a luminometer.

16. A method of identifying mammary progenitor cells in a population of mammary cells, the method comprising:
a) introducing into the population of mammary cells an expression cassette comprising (i) a first nucleotide sequence encoding a reporter, and (ii) a second nucleotide sequence complementary to about 12-25 contiguous nucleotides of miR-205 or miR-22 in a cell inhibits expression of the reporter in the cell, wherein the presence of miR-205 or miR-22 in a cell inhibits expression of the reporter in the cell; and,
b) identifying cells that do not express the reporter;
thereby identifying mammary progenitor cells.

17. The method of claim 16, wherein the expression cassette comprises a tissue-specific promoter, a developmental stage specific promoter, or an inducible promoter.

18. The method of claim 16, wherein cells not expressing the reporter are identified using a luminometer.
